(19) United States
(12) Patent Application Publication
(10) Pub. No.: US 2009/0162911 A1
(43) Pub. Date: Jun. 25, 2009

(54) STRAIN FOR BUTANOL PRODUCTION

(75) Inventors: Robert A. Larossa, Chadds Ford, PA (US); Dana R. Smulski, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY

(21) Appl. No.: 12/330,531

(22) Filed: Dec. 9, 2008

Related U.S. Application Data
(60) Provisional application No. 61/015,712, filed on Dec. 21, 2007.
(60) Provisional application No. 61/015,721, filed on Dec. 21, 2007.

(51) Int. Cl. C12P 7/26; C12N 1/21; C12N 15/87; C12P 7/16

(52) U.S. Cl. 435/148; 435/252.33; 435/440; 435/160

(57) ABSTRACT

Using screening of transposon random insertion mutants, genes involved in a complex that is a three-component proton motive force-dependent multidrug efflux system were found to be involved in E. coli cell response to butanol. Reduced production of the AcrA and/or AcrB proteins of the complex confers increased butanol tolerance. E. coli strains with reduced AcrA or AcrB production and having a butanol or 2-butanone biosynthetic pathway are useful for production of butanol or 2-butanone.

This application claims the benefit of U.S. Applications 61/015,712 and 61/015,721, both filed Dec. 21, 2007, both now pending.

### FIELD OF INVENTION

The invention relates to the fields of microbiology and genetic engineering. More specifically, bacterial genes involved in tolerance to butanol were identified. Bacterial strains with reduced expression of the identified genes were found to have improved growth yield in the presence of butanol.

### BACKGROUND OF INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Methods for the chemical synthesis of butanols are known. For example, 1-butanol may be produced using the Oxo process, the Reppe process, or the hydrogenation of crotonaldehyde (Ullmann's Encyclopedia of Industrial Chemistry, 6^{th} edition, 2003, Wiley-VCHVerlag GmbH and Co., Weinheim, Germany, Vol. 5, pp. 716-719). 2-Butanol may be produced using n-butene hydration (Ullmann's Encyclopedia of Industrial Chemistry, 6^{th} edition, 2003, Wiley-VCHVerlag GmbH and Co., Weinheim, Germany, Vol. 5, pp. 716-719). Additionally, isobutanol may be produced using Oxo synthesis, catalytic hydrogenation of carbon monoxide (Ullmann's Encyclopedia of Industrial Chemistry, 6^{th} edition, 2003, Wiley-VCHVerlag GmbH and Co., Weinheim, Germany, Vol. 5, pp. 716-719) or Guerbet condensation of methanol with n-propanol (Carlini et al., J. Molec. Catal. A:Chem. 220:215-220 (2004)). These processes use starting materials derived from petrochemicals, are generally expensive, and are not environmentally friendly.

Methods of producing butanol by fermentation are also known, where the most popular process produces a mixture of acetone, 1-butanol and ethanol and is referred to as the ABE processes (Blaschek et al., U.S. Pat. No. 6,358,717). Acetone-butanol-ethanol (ABE) fermentation by Clostridium acetobutylicum is one of the oldest known industrial fermentations, and the pathways and genes responsible for the production of these solvents have been reported (Girbal et al., Trends in Biotechnology 16:11-16 (1998)). Additionally, recombinant microbial production hosts expressing a 1-butanol biosynthetic pathway (Donaldson et al., copending and commonly owned U.S. Patent Application Publication No. US20080182308A1), a 2-butanol biosynthetic pathway (Donaldson et al., copending and commonly owned U.S. Patent Application Publication Nos. US20070259410A1 and US20070292927A1), and an isobutanol biosynthetic pathway (Maggio-Hall et al., copending and commonly owned U.S. Patent Publication No. US 20070092957) have been described. However, biological production of butanols is believed to be limited by butanol toxicity to the host microorganism used in the fermentation.

In addition, 2-butanone is a valuable compound that can be produced by fermentation using microorganisms. 2-Butanone, also referred to as methyl ethyl ketone (MEK), is a widely used solvent and is the most important commercially produced ketone, after acetone. It is used as a solvent for paints, resins, and adhesives, as well as a selective extractant and activator of oxidative reactions. In addition, it has been shown that substantially pure 2-butanone can be converted to 2-butanol by reacting with hydrogen in the presence of a catalyst (Nystrom, R. F. and Brown, W. G. (J. Am. Chem. Soc. (1947) 69:1198). 2-butanone can be made by omitting the last step of the 2-butanol biosynthetic pathway (Donaldson et al., copending and commonly owned U.S. Patent Application Publication Nos. US20070259410A1 and US 20070292927A1). Production of 2-butanone would be enhanced by using microbial host strains with improved tolerance as fermentation biocatalysts.

Strains of Clostridium that are tolerant to 1-butanol have been isolated by chemical mutagenesis (Jain et al. U.S. Pat. No. 5,192,673; and Blaschek et al. U.S. Pat. No. 6,358,717), overexpression of certain classes of genes such as those that express stress response proteins (Papoutsakis et al. U.S. Pat. No. 6,960,465; and Tomas et al., Appl. Environ. Microbiol. 69(8):4951-4965 (2003)), and by serial enrichment (Quratulain et al., Folia Microbiologica (Prague) 40(5):467-471 (1995); and Soucaille et al., Current Microbiology 14(5):295-299 (1987)). Desmond et al. (Appl. Environ. Microbiol. 70(10):5929-5936 (2004)) report that overexpression of GroESL, two stress responsive proteins, in Lactococcus lactis and Lactobacillus paracasei produced strains that were able to grow in the presence of 0.5% volume/volume (v/v) [0.4% weight/volume (w/v)] 1-butanol. Additionally, the isolation of 1-butanol tolerant strains from estuary sediment (Sardessai et al., Current Science 82(6):622-623 (2002)) and from activated sludge (Bieszkiewicz et al., Acta Microbiologica Polonica 36(3):259-265 (1987)) has been described. Additionally some Lactobacillus sp are known to be tolerant to ethanol (see for example, Couto, Pina and Hogg Biotechnology. Letter 19: 487-490). Ingram and Burke (1984) Adv. Microbial. Physiol 25: 253-300. However, for most bacteria described in the art, growth is highly inhibited at low concentrations of 1-butanol. Moreover butanol is much more toxic than ethanol and mechanisms that affect the ethanol tolerance of E. coli have not been found to affect the butanol response.

There is a need, therefore, for butanol or 2-butanone producing bacterial host strains that are more tolerant to these chemicals as well as methods of producing butanols or 2-butanone using bacterial host strains that are more tolerant to these chemicals.

### SUMMARY OF THE INVENTION

The invention provides a recombinant Escherichia coli host which produces butanol or 2-butanone and comprises a genetic modification that results in reduced production of AcrA, AcrB, or both AcrA and AcrB, which are two endogenous proteins known to be components of a multidrug efflux pump. Such cells have an increased tolerance to butanol or 2-butanone as compared with cells that lack the genetic modification. Host cells of the invention may produce butanol or 2-butanone naturally or may be engineered to do so via an engineered pathway.

Accordingly, the invention provides A recombinant Escherichia coli cell producing butanol or 2-butanone said E. coli cell comprising at least one genetic modification which reduces production of a protein selected from the group consisting of AcrA and AcrB.

In another embodiment the invention provides a process for generating the E. coli host cell of claim 1 comprising:
a) providing a recombinant bacterial host cell producing butanol or 2-butanone; and
b) creating at least one genetic modification which redues production of AcrA or AcrB, or both AcrA and AcrB proteins.

In another embodiment the invention provides a process for production of butanol or 2-butanone from a recombinant E. coli cell comprising:
(a) providing a recombinant E. coli cell which
   1) produces butanol or 2-butanone and
   2) comprises at least one genetic modification which reduces production of AcrA or AcrB, or both AcrA and AcrB; and
(b) culturing the strain of (a) under conditions wherein butanol or 2-butanone is produced.

### BRIEF DESCRIPTION FIGURES AND SEQUENCE DESCRIPTIONS

The various embodiments of the invention can be more fully understood from the following detailed description, the figures, and the accompanying sequence descriptions, which form a part of this application.

FIG. 1 shows a graph of the difference between 4 hour and 2 hour growth time points for an acrB insertion mutant in different concentrations of 1-butanol.

FIG. 2 shows a graph of percent growth inhibition by different concentrations of 1-butanol in an acrB insertion mutant strain.

FIG. 3 shows a graph of growth of an acrB transposon insertion line (A) and EC100 (B) in different concentrations of 1-butanol.

FIG. 4 shows a graph of growth of the constructed acrB rpoZ double mutant, acrB marker deletion and rpoZ marker insertion lines and the control in the absence of 1-butanol.

FIG. 5 shows graphs of growth in 0, 0.4% or 0.6% 1-butanol of the constructed acrB marker deletion line (A; DPD1876) and constructed acrB rpoZ double mutant line (B; DPD1899).

FIG. 6 shows a graph of the fractional growth of the constructed acrB rpoZ double mutant, acrB marker deletion and rpoZ marker insertion line and the control in different concentrations of 1-butanol.

FIG. 7 shows a graph of percent improvement in growth of the acrB transposon mutant line as compared to the parental strain in various concentrations of butanols and MEK.

FIG. 8 shows a graph of percent improvement in growth of the acrA and acrB transposon mutant lines as compared to the parental strain in two concentrations of 2-butanol (A) and isobutanol (B).

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
Summary of Gene and Protein SEQ ID Numbers
for 1-Butanol Biosynthetic Pathway

| | SEQ ID NO: | SEQ ID NO: |
|---|---|---|
| Description | Nucleic acid | Peptide |
| Acetyl-CoA acetyltransferase thlA from | 1 | 2 |
| Clostridium acetobutylicum ATCC 824 | | |
| Acetyl-CoA acetyltransferase thlB from | 3 | 4 |
| Clostridium acetobutylicum ATCC 824 | | |
| 3-Hydroxybutyryl-CoA dehydrogenase | 5 | 6 |
| from Clostridium acetobutylicum ATCC | | |
| 824 | | |
| Crotonase from Clostridium | 7 | 8 |
| acetobutylicum ATCC 824 | | |
| Putative trans-enoyl CoA reductase from | 9 | 10 |
| Clostridium acetobutylicum ATCC 824 | | |
| Butyraldehyde dehydrogenase from | 11 | 12 |
| Clostridium beijerinckii NRRL B594 | | |
| 1-Butanol dehydrogenase bdhB from | 13 | 14 |
| Clostridium acetobutylicum ATCC 824 | | |
| 1-Butanol dehydrogenase bdhA from | 15 | 16 |
| Clostridium acetobutylicum ATCC 824 | | |

TABLE 2
Summary of Gene and Protein SEQ ID Numbers
for 2-Butanol Biosynthetic Pathway

| | SEQ ID NO: | SEQ ID NO: |
|---|---|---|
| Description | Nucleic acid | Peptide |
| budA, acetolactate decarboxylase from | 17 | 18 |
| Klebsiella pneumoniae ATCC 25955 | | |
| budB, acetolactate synthase from | 19 | 20 |
| Klebsiella pneumoniae ATCC 25955 | | |
| budC, butanediol dehydrogenase from | 21 | 22 |
| Klebsiella pneumoniae IAM1063 | | |
| pddA, butanediol dehydratase alpha | 23 | 24 |
| subunit from Klebsiella oxytoca ATCC | | |
| 8724 | | |
| pddB, butanediol dehydratase beta | 25 | 26 |
| subunit from Klebsiella oxytoca ATCC | | |
| 8724 | | |
| pddC, butanediol dehydratase gamma | 27 | 28 |
| subunit from Klebsiella oxytoca ATCC | | |
| 8724 | | |
| sadH, 2-butanol dehydrogenase from | 29 | 30 |
| Rhodococcus ruber 219 | | |

TABLE 3
Summary of Gene and Protein SEQ ID Numbers
for Isobutanol Biosynthetic Pathway

| | SEQ ID NO: | SEQ ID NO: |
|---|---|---|
| Description | Nucleic acid | Peptide |
| Klebsiella pneumoniae budB | 19 | 20 |
| (acetolactate synthase) | | |
| E. coli ilvC (acetohydroxy acid | 31 | 32 |
| reductoisomerase) | | |
| E. coli ilvD (acetohydroxy acid | 33 | 34 |
| dehydratase) | | |
| Lactococcus lactis kivD (branched-chain | 35 | 36 |
| α-keto acid decarboxylase), codon | | |
| optimized | | |
| E. coli yqhD (branched-chain alcohol | 37 | 38 |
| dehydrogenase) | | |

TABLE 4
Gene and Protein SEQ ID Numbers for E. coli butanol
tolerance target genes

| | SEQ ID NO: | SEQ ID NO: |
|---|---|---|
| Description | Nucleic acid | Peptide |
| E. coli K12 acrA | 39 | 40 |
| E. coli K12 acrB | 41 | 42 |
| E. coli o157:h7 acrA | 43 | 44 |
| E. coli CFT073 acrA | 45 | 46 |
| E. coli UTI89 acrA | 47 | 48 |
| E. coli o157:h7 acrB | 49 | 50 |
| E. coli CFT073 acrB | 51 | 52 |
| E. coli UTI89 acrB | 53 | 54 |
| E. coli K12 spoT | 55 | 56 |
| E. coli o157:h7 spoT | 57 | 58 |
| E. coli CFT073 spoT | 59 | 60 |
| E. coli UTI89 spoT | 61 | 62 |
| E. coli K12 relA | 63 | 64 |
| E. coli o157:h7 relA | 65 | 66 |
| E. coli CFT073 relA | 67 | 68 |
| E. coli UTI89 relA | 69 | 70 |

SEQ ID NO:71 is the nucleotide sequence of the acrAB operon promoter region.

SEQ ID NOs:72 and 73 are sequencing primers that read outward from each end of the transposon used to make knockout mutations for butanol screening.

### DETAILED DESCRIPTION OF THE INVENTION

The present invention provides a recombinant E. coli host which produces butanol or 2-butanone and comprises a genetic modification that results in reduced production of AcrA, AcrB, or both AcrA and AcrB. Such cells have an increased tolerance to butanol or 2-butanone as compared with cells that lack the genetic modification. A tolerant bacterial strain of the invention has at least one genetic modification that causes reduced production of AcrA and/or AcrB. Host cells of the invention may produce butanol or 2-butanone naturally or may be engineered to do so via an engineered pathway.

Butanol produced using the present strains may be used as an alternative energy source to fossil fuels, and 2-butanone may be used as a solvent or may be chemically converted to 2-butanol. Fermentive production of butanol and 2-butanone results in less pollutants than typical petrochemical synthesis.

The following abbreviations and definitions will be used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “butanol” as used herein, refers to 1-butanol, 2-butanol, isobutanol, or mixtures thereof.

The terms “butanol tolerant bacterial strain” and “tolerant” when used to describe a modified bacterial strain of the invention, refers to a modified bacterium that shows better growth in the presence of butanol than the parent strain from which it is derived. 2-butanone tolerance is used similarly.

The term “butanol biosynthetic pathway” refers to an enzyme pathway to produce 1-butanol, 2-butanol, or isobutanol.

The term “1-butanol biosynthetic pathway” refers to an enzyme pathway to produce 1-butanol from acetyl-coenzyme A (acetyl-CoA).

The term “2-butanol biosynthetic pathway” refers to an enzyme pathway to produce 2-butanol from pyruvate.

The term “isobutanol biosynthetic pathway” refers to an enzyme pathway to produce isobutanol from pyruvate.

The term “2-butanone biosynthetic pathway” refers to an enzyme pathway to produce 2-butanone from pyruvate.

The term “acetyl-CoA acetyltransferase” refers to an enzyme that catalyzes the conversion of two molecules of acetyl-CoA to acetoacetyl-CoA and coenzyme A (CoA). Preferred acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego]; although, enzymes with a broader substrate range (E.C. 2.3.1.16) will be functional as well. Acetyl-CoA acetyltransferases are available from a number of sources, for example, Escherichia coli (GenBank Nos: NP_{—}416728, NC_{—}000913; NCBI (National Center for Biotechnology Information) amino acid sequence, NCBI nucleotide sequence), Clostridium acetobutylicum (GenBank Nos: NP_{—}349476.1 (SEQ ID NO:2), NC_{—}003030; NP_{—}149242 (SEQ ID NO:4), NC_{—}001988), Bacillus subtilis (GenBank Nos: NP_{—}390297, NC_{—}000964), and Saccharomyces cerevisiae (GenBank Nos: NP_{—}015297, NC_{—}001148).

The term “3-hydroxybutyryl-CoA dehydrogenase” refers to an enzyme that catalyzes the conversion of acetoacetyl-CoA to 3-hydroxybutyryl-CoA. 3-Hydroxybutyryl-CoA dehydrogenases may be reduced nicotinamide adenine dinucleotide (NADH)-dependent, with a substrate preference for (S)-3-hydroxybutyryl-CoA or (R)-3-hydroxybutyryl-CoA and are classified as E.C. 1.1.1.35 and E.C. 1.1.1.30, respectively. Additionally, 3-hydroxybutyryl-CoA dehydrogenases may be reduced nicotinamide adenine dinucleotide phosphate (NADPH)-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-Hydroxybutyryl-CoA dehydrogenases are available from a number of sources, for example, C. acetobutylicum (GenBank NOs: NP_{—}349314 (SEQ ID NO:6), NC_{—}003030), B. subtilis (GenBank NOs: AAB09614, U29084), Ralstonia eutropha (GenBank NOs: ZP_{—}0017144, NZ_AADY01000001, Alcaligenes eutrophus (GenBank NOs: YP_{—}294481, NC_{—}007347), and A. eutrophus (GenBank NOs: P14697, J04987).

The term “crotonase” refers to an enzyme that catalyzes the conversion of 3-hydroxybutyryl-CoA to crotonyl-CoA and H₂O. Crotonases may have a substrate preference for (S)-3-hydroxybutyryl-CoA or (R)-3-hydroxybutyryl-CoA and are classified as E.C. 4.2.1.17 and E.C. 4.2.1.55, respectively. Crotonases are available from a number of sources, for example, E. coli (GenBank NOs: NP_{—}415911 (SEQ ID NO:8), NC_{—}000913), C. acetobutylicum (GenBank NOs: NP_{—}349318, NC_{—}003030), B. subtilis (GenBank NOs: CAB13705, Z99113), and Aeromonas caviae (GenBank NOs: BAA21816, D88825).

The term “butyryl-CoA dehydrogenase”, also called trans-enoyl CoA reductase, refers to an enzyme that catalyzes the conversion of crotonyl-CoA to butyryl-CoA. Butyryl-CoA dehydrogenases may be NADH-dependent or NADPH-dependent and are classified as E.C. 1.3.1.44 and E.C. 1.3.1.38, respectively. Butyryl-CoA dehydrogenases are available from a number of sources, for example, C. acetobutylicum (GenBank NOs: NP_{—}347102 (SEQ ID NO:10), NC_{—}003030), Euglena gracilis (GenBank NOs: □5EU90, AY741582), Streptomyces collinus (GenBank NOs: AAA92890, U37135), and Streptomyces coelicolor (GenBank NOs: CAA22721, AL939127).

The term “butyraldehyde dehydrogenase” refers to an enzyme that catalyzes the conversion of butyryl-CoA to butyraldehyde, using NADH or NADPH as cofactor. Butyraldehyde dehydrogenases with a preference for NADH are known as E.C. 1.2.1.57 and are available from, for example, Clostridium beijerinckii (GenBank NOs: AAD31841 (SEQ ID NO:12), AF157306) and C. acetobutylicum (GenBank NOs: NP_{—}149325, NC_{—}001988).

The term “1-butanol dehydrogenase” refers to an enzyme that catalyzes the conversion of butyraldehyde to 1-butanol. 1-butanol dehydrogenases are a subset of the broad family of alcohol dehydrogenases. 1-butanol dehydrogenase may be NADH- or NADPH-dependent. 1-butanol dehydrogenases are available from, for example, C. acetobutylicum (GenBank NOs: NP_{—}149325, NC_{—}001988; NP_{—}349891 (SEQ ID NO:14), NC_{—}003030; and NP_{—}349892 (SEQ ID NO:16), NC_{—}003030) and E. coli (GenBank NOs: NP_{—}417484, NC_{—}000913).

The term “acetolactate synthase”, also known as “acetohydroxy acid synthase”, refers to a polypeptide (or polypeptides) having an enzyme activity that catalyzes the conversion of two molecules of pyruvic acid to one molecule of alpha-acetolactate. Acetolactate synthase, known as EC 2.2.1.6 [formerly 4.1.3.18] (Enzyme Nomenclature 1992, Academic Press, San Diego) may be dependent on the cofactor thiamin pyrophosphate for its activity. Suitable acetolactate synthase enzymes are available from a number of sources, for example, Bacillus subtilis (GenBank Nos: AAA22222 NCBI (National Center for Biotechnology Information) amino acid sequence, L04470 NCBI nucleotide sequence), Klebsiella terrigena (GenBank Nos: AAA25055, L04507), and Klebsiella pneumoniae (GenBank Nos: AAA25079 (SEQ ID NO:20), M73842 (SEQ ID NO:19).

The term “acetolactate decarboxylase” refers to a polypeptide (or polypeptides) having an enzyme activity that catalyzes the conversion of alpha-acetolactate to acetoin. Acetolactate decarboxylases are known as EC 4.1.1.5 and are available, for example, from Bacillus subtilis (GenBank Nos: AAA22223, L04470), Klebsiella terrigena (GenBank Nos: AAA25054, L04507) and Klebsiella pneumoniae (SEQ ID NO:18 (amino acid) SEQ ID NO:17 (nucleotide)).

The term “butanediol dehydrogenase” also known as “acetoin reductase” refers to a polypeptide (or polypeptides) having an enzyme activity that catalyzes the conversion of acetoin to 2,3-butanediol. Butanediol dehydrogenases are a subset of the broad family of alcohol dehydrogenases. Butanediol dehydrogenase enzymes may have specificity for production of R- or S-stereochemistry in the alcohol product. S-specific butanediol dehydrogenases are known as EC 1.1.1.76 and are available, for example, from Klebsiella pneumoniae (GenBank Nos: BBA13085 (SEQ ID NO:22), D86412. R-specific butanediol dehydrogenases are known as EC 1.1.1.4 and are available, for example, from Bacillus cereus (GenBank Nos. NP_{—}830481, NC_{—}004722; AAP07682, AE017000), and Lactococcus lactis (GenBank Nos. AAK04995, AE006323).

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone, also known as methyl ethyl ketone (MEK). Butanediol dehydratase may utilize the cofactor adenosyl cobalamin. Adenosyl cobalamin-dependent enzymes are known as EC 4.2.1.28 and are available, for example, from Klebsiella oxytoca (GenBank Nos: BAA08099 (alpha subunit) (SEQ ID NO:24), BAA08100 (beta subunit) (SEQ ID NO:26), and BBA08101 (gamma subunit) (SEQ ID NO:28), (Note all three subunits are required for activity), D45071).

The term “2-butanol dehydrogenase” refers to a polypeptide (or polypeptides) having an enzyme activity that catalyzes the conversion of 2-butanone to 2-butanol. 2-butanol dehydrogenases are a subset of the broad family of alcohol dehydrogenases. 2-butanol dehydrogenase may be NADH- or NADPH-dependent. The NADH-dependent enzymes are known as EC 1.1.1.1 and are available, for example, from Rhodococcus ruber (GenBank Nos: CAD36475 (SEQ ID NO:30), AJ491307 (SEQ ID NO:29)). The NADPH-dependent enzymes are known as EC 1.1.1.2 and are available, for example, from Pyrococcus furiosus (GenBank Nos: AAC25556, AF013169).

The term “acetohydroxy acid isomeroreductase” or “acetohydroxy acid reductoisomerase” refers to an enzyme that catalyzes the conversion of acetolactate to 2,3-dihydroxyisovalerate using NADPH (reduced nicotinamide adenine dinucleotide phosphate) as an electron donor. Preferred acetohydroxy acid isomeroreductases are known by the EC number 1.1.1.86 and sequences are available from a vast array of microorganisms, including, but not limited to, Escherichia coli (GenBank Nos: NP_{—}418222 (SEQ ID NO:32), NC_{—}000913 (SEQ ID NO:31)), Saccharomyces cerevisiae (GenBank Nos: NP_{—}013459, NC_{—}001144), Methanococcus maripaludis (GenBank Nos: CAF30210, BX957220), and Bacillus subtilis (GenBank Nos: CAB14789, Z99118).

The term “acetohydroxy acid dehydratase” refers to an enzyme that catalyzes the conversion of 2,3-dihydroxyisovalerate to α-ketoisovalerate. Preferred acetohydroxy acid dehydratases are known by the EC number 4.2.1.9. These enzymes are available from a vast array of microorganisms, including, but not limited to, E. coli (GenBank Nos: YP_{—}026248 (SEQ ID NO:34), NC_{—}000913 (SEQ ID NO:33)), S. cerevisiae (GenBank Nos: NP_{—}012550, NC_{—}001142), M. maripaludis (GenBank Nos: CAF29874, BX957219), and B. subtilis (GenBank Nos: CAB14105, Z99115).

The term “branched-chain α-keto acid decarboxylase” refers to an enzyme that catalyzes the conversion of α-ketoisovalerate to isobutyraldehyde and CO₂. Preferred branched-chain α-keto acid decarboxylases are known by the EC number 4.1.1.72 and are available from a number of sources, including, but not limited to, Lactococcus lactis (GenBank Nos: AAS49166, AY548760; CAG34226 (SEQ ID NO:36), AJ746364, Salmonella typhimurium (GenBank Nos: NP_{—}461346, NC_{—}003197), and Clostridium acetobutylicum (GenBank Nos: NP_{—}149189, NC_{—}001988).

The term “branched-chain alcohol dehydrogenase” refers to an enzyme that catalyzes the conversion of isobutyraldehyde to isobutanol. Preferred branched-chain alcohol dehydrogenases are known by the EC number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as electron donor and are available from a number of sources, including, but not limited to, S. cerevisiae (GenBank Nos: NP_{—}010656, NC_{—}001136; NP_{—}014051, NC_{—}001145), E. coli (GenBank Nos: NP_{—}417484 (SEQ ID NO:38), NC_{—}000913 (SEQ ID NO:37)), and C. acetobutylicum (GenBank Nos: NP_{—}349892, NC_{—}003030).

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein the term “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” and “vector” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation vector” refers to a specific vector containing a foreign gene and having elements in addition to the foreign gene that facilitates transformation of a particular host cell.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “(p)ppGpp” refers to either ppGpp or pppGpp, or a combination of both compounds.

The term “re/A” refers to a gene that encodes a RelA protein which is a mono-functional enzyme with GTP pyrophosphokinase activity (EC 2.7.6.5), for synthesis of (p)ppGpp. Although in the literature some genes encoding enzymes with (p)ppGpp synthesis and degradation activities are called re/A, herein these will be referred to as spoT instead of re/A.

The term “spoT” refers to a gene that encodes a SpoT protein, which is a bi-functional enzyme with both GTP pyrophosphokinase, (EC 2.7.6.5) activity for synthesis of (p)ppGpp, and ppGpp pyrophosphohydrolase (EC3.1.7.2) activity for degradation of (p)ppGpp. The related RelA and SpoT proteins and their encoding genes are distinguished by both enzyme activities and domain architectures as described below.

The term “RelA/SpoT” domain will refer to a portion of the SpoT or RelA proteins that may be used to identity SpoT or RelA homologs.

As used herein “TGS domain” will refer to a portion of the SpoT or RelA protein that may be used to identity SpoT and RelA homologs. The TGS domain is named after ThrRS, GTPase, and SpoT and has been detected at the amino terminus of the uridine kinase from the spirochaete Treponema pallidum. TGS is a small domain that consists of ˜50 amino acid residues and is predicted to possess a predominantly beta-sheet structure. Its presence in two types of regulatory proteins (the GTPases and guanosine polyphosphate phosphohydrolases/synthetases) suggests that it has a nucleotide binding regulatory role. The TGS domain is not unique to the SpoT or RelA protein, however, in combination with the presense of the HD domain and the SpoT/RelA domain it is diagnostic for a protein having SpoT function. In combination with the SpoT/RelA domain, the TGS domain is diagnostic for a protein having RelA function.

The term “HD domain” refers to an amino acid motif that is associated with a superfamily of metal-dependent phosphohydrolases that includes a variety of uncharacterized proteins and domains associated with nucleotidyltransferases and helicases from bacteria, archaea, and eukaryotes (Yakunin et al., J. Biol. Chem., Vol. 279, Issue 35, 36819-36827, Aug. 27, 2004). The HD domain is not unique to the SpoT protein, however in combination with the SpoT/RelA domain and the TGS domain, it may be used to identify SpoT proteins according to the methods described herein.

The term “dksA” refers to a gene that encodes the DksA protein, which binds directly to RNA polymerase affecting transcript elongation and augmenting the effect of the alarmone ppGpp on transcription initiation.

The term “efflux pump” refers to a set of proteins that actively transport a compound from the cytoplasm out into the medium.

Herein, a modified acrA or acrB strain refers to a genetically modified strain with reduced or no AcrA and/or AcrB protein production.

Standard recombinant DNA and molecular cloning techniques used here are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987.

### Screening for Butanol Tolerance: Involvement of AcrA and AcrB

The invention relates to the discovery that events that disrupt the production of AcrB in an E. coli cell have the unexpected effect of rendering the cell more tolerant to butanols. The discovery came out of screening studies for genetic mutations that affected butanol tolerance. In those studies, E. coli cells were subjected to random mutagenesis and then screened for altered tolerance to butanol. Those mutants showing higher butanol tolerance were analyzed and the affected genes identified. The modified gene leading to butanol tolerance in a mutant may be identified by methods as described herein in Example 2 for a transposon insertion strain, or by directed genome sequencing of candidate genes in the case of chemical mutagenesis. If the bacterial cell has a means of genetic exchange, then genetic crosses may be performed to verify that the effect is due to the observed alteration in the genome.

These studies indicated that disruptions in AcrB protein production correlated to an increase in butanol tolerance. The E. coli AcrB protein (SEQ ID NO:42; coding region: SEQ ID NO:41) is one protein in a complex that is a three-component proton motive force-dependent multidrug efflux system. The other components are proteins AcrA (SEQ ID NO:40; coding region: SEQ ID NO:39) and TolC. The complex is a major contributor to the intrinsic resistance of E. coli to solvents, dyes and detergents as well as lipophilic antibiotics including novobiocin, erythromycin, fusidic acid and cloxacillin. Overexpression of the complex components results in resistance to multiple antimicrobial agents, including the common antibiotics tetracycline and chloramphenicol. Thus it is surprising that reduced expression of the AcrB protein of the multidrug efflux complex results in increased butanol tolerance. Applicants also found that reduced expression of the complex component acrA increases tolerance, but reduced expression of the complex component TolC did not.

### Genetic Modification to Reduce AcrA or AcrB Expression

As noted above, mutations that affect production of the AcrA protein or AcrB protein of E. coli cells have been associated herein with an increase in tolerance of the cell to butanol. Accordingly the invention provides an E. coli comprising at least one genetic modification which reduces production of AcrA or AcrB.

In the present E. coli cells, a modification is engineered that results in decreased expression of the AcrA or AcrB protein, or both AcrA and AcrB proteins, to increase butanol tolerance. Many methods for genetic modification are known to one skilled in the art which may be used, including directed gene modification as well as random genetic modification followed by screening. Typically used random genetic modification methods (reviewed in Miller, J. H. (1992) A Short Course in Bacterial Genetics. Cold Spring Harbor Press, Plainview, N.Y.) include spontaneous mutagenesis, mutagenesis caused by mutator genes, chemical mutagenesis, irradiation with UV or X-rays, and transposon insertion. Transposons have been introduced into bacteria in a variety of ways including:
1. phage-mediated transduction. This has been used in both species specific and cross-species contexts.
2. conjugation. Again this can be between members of the same or different species.
3. Transformation. Chemically aided and electric shock mediated uptake of DNA can be used.
   In these cases the transposon expresses a transposase in the recipient that catalyzes gene hopping from the incoming DNA to the recipient genome. The transposon DNA can be naked, incorporated in a phage or plasmid nucleic acid or complexed with a transposase. Most often the replication and/or maintenance of the incoming DNA containing the transposon is prevented, such that genetic selection for a marker on the transposon (most often antibiotic resistance) insures that each recombinant is the result of movement of the transposon from the entering DNA molecule to the recipient genome. An alternative method is one in which transposition is carried out with chromosomal DNA, fragments thereof, or a fragment thereof in vitro, and then the novel insertion allele that has been created is introduced into a recipient cell where it replaces the resident allele by homologous recombination. Transposon insertion may be performed as described in Kleckner and Botstein ((1977) J. Mol. Biol. 116:125-159), or as indicated above via any number of derivative methods, or as described in Example 1 using the Transposome™ system (Epicentre; Madison, Wis.).

Chemical mutagenesis may be performed as described in Miller (Unit 4 of Miller J H (1992) A Short Course in Bacterial Genetics, Cold Spring Harbor Laboratory Press, pp 81-211). Collections of modified cells produced from these processes may be screened either for butanol tolerance, as described in Example 1 herein, or for reduced expression of AcrA or AcrB using protein or RNA analysis as known to one skilled in the art.

When strains are selected following screening for butanol tolerance, the selected strains are then assayed for reduced AcrA or AcrB expression, and/or the modified gene is determined. The modified gene leading to butanol tolerance may be identified as described herein in Example 2 for a transposon insertion strain, or by directed genome sequencing of candidate genes in the case of chemical mutagenesis. If the organism has a means of genetic exchange then genetic crosses may be performed to verify that the effect is due to the observed alteration in the genome.

In addition, any directed genetic modification method known by one skilled in the art for reducing the expression of a functional protein may be used to make at least one modification to reduce AcrA or AcrB production in the present E. coli cells. Many methods involve modifications to the encoding gene. Target coding sequences for modifying AcrA and AcrB production are SEQ ID NO: 39 and SEQ ID NO: 41, respectively. These sequences are from the K12 strain of E. coli. Sequences encoding AcrA and AcrB from other strains of E. coli are readily recognized by one skilled in the art, having only few variations with sequence identities of at least about 96%, 97%, 98%, or 99% and are targets for modification in their host strains. For example, acrA coding regions and AcrA proteins, respectively, for different E. coli strains are as follows: E. coli o157:h7 SEQ ID NO:43 and SEQ ID NO:44; E. coli CFT073 SEQ ID NO:45 and SEQ ID NO:46; E. coli UT189 SEQ ID NO:47 and SEQ ID NO:48. For example, acrB coding regions and AcrB proteins, respectively, for different E. coli strains are as follows: E. coli o157:h7 SEQ ID NO:49 and SEQ ID NO:50; E. coli CFT073 SEQ ID NO:51 and SEQ ID NO:52; E. coli UT189 SEQ ID NO:53 and SEQ ID NO:54.

Genetic modification methods include, but are not limited to, deletion of the entire gene or a portion of the gene encoding AcrA or AcrB, inserting a DNA fragment into the acrA or acrB gene (in either the promoter or coding region) so that the protein is not expressed or expressed at lower levels, introducing a mutation into the acrA or acrB coding region which adds a stop codon or frame shift such that a functional protein is not expressed, and introducing one or more mutations into the acrA or acrB coding region to alter amino acids so that a non-functional or a less functional protein is expressed. In addition, acrA or acrB expression may be blocked by expression of an antisense RNA or an interfering RNA, and constructs may be introduced that result in cosuppression. In addition the synthesis of or stability of the transcript may be lessened by mutation. Similarly the efficiency by which a protein is translated from mRNA may be modulated by mutation. All of these methods may be readily practiced by one skilled in the art making use of the known sequences encoding AcrA or AcrB proteins. DNA sequences surrounding the acrA or acrB coding sequences are also useful in some modification procedures and are available for E. coli in the complete genome sequence of the K12 strain: GenBank Accession #U00096.2.

In particular, DNA sequences surrounding the acrA or acrB coding sequence are useful for modification methods using homologous recombination. For example, in this method acrB gene flanking sequences are placed bounding a selectable marker gene to mediate homologous recombination whereby the marker gene replaces the acrB gene. Also partial acrB gene sequences and acrB flanking sequences bounding a selectable marker gene may be used to mediate homologous recombination whereby the marker gene replaces a portion of the acrB gene. In addition, the selectable marker may be bounded by site-specific recombination sites, so that following expression of the corresponding site-specific recombinase, the resistance gene is excised from the acrB gene without reactivating the latter. The site-specific recombination leaves behind a recombination site which disrupts expression of the AcrB protein. The homologous recombination vector may be constructed to also leave a deletion in the acrB gene following excision of the selectable marker, as is well known to one skilled in the art. Moreover, promoter replacement methods may be used to exchange the endogenous transcriptional control elements allowing another means to modulate expression such as described in Yuan et al. (Metab Eng. (2006) 8:79-90).

Another means of reducing acrA and acrB expression is to fuse the promoter of the acrAB operon (SEQ ID NO:71) to the lac operon (Silhavy, Berman, and Enquist (1984) Experiments with Gene Fusions. Cold Spring Harbor Laboratory, Cold Spring Harbor, N.Y.) and use the well described selections and screens to obtain mutants with decreased expression driven from the promoter (Beckwith (1978)/ac: The Genetic System, p:11-30. In J. Miller and W. Reznikoff (ed.), The Operon. Cold Spring Harbor Laboratory, Cold Spring Harbor, N.Y.; Miller (1972) Experiments in molecular genetics. Cold Spring Harbor Laboratory Press, Cold Spring Harbor, N.Y.; Miller (1992) A Short Course in Bacterial Genetics. Cold Spring Harbor Press, Plainview, N.Y.). The lower activity promoter is then used to replace the endogenous promoter, typically using homologous recombination, to decrease expression of acrA and acrB, since these two coding regions are in an operon (acrAB). Moreover not only can cis-acting promoter down mutations be expected to satisfy the criterion of lowering acrAB expression, but isolation of super-repressing variants (Bourgeois and Jobe (1970) Superrepressors of thelac operon, p. 325-341 In J. Beckwith and D. Zipser (ed.), The lactose operon. Cold Spring Harbor Laboratory, NY) of the adjacent acrR gene would also lower the titer of AcrA and AcrB, since AcrR is a transcriptional repressor of acrAB (Ma et al. (1996) Mol. Microbiol. 19:101-112).

The sequence for the promoter of the acrAB operon given as SEQ ID NO:71 is for the E. coli K12 strain. One skilled in the art will readily recognize the promoter of the acrAB operon in other strains of E. coli, which may include sequence variations, due to its location 5′ to the coding region for AcrA.

### Butanol Tolerance of Reduced AcrA or AcrB Strain

An E. coli strain of the present invention genetically modified for reduced expression of AcrA and/or acrB has improved tolerance to butanol. The tolerance of reduced AcrA and/or AcrB strains may be assessed by assaying their growth in concentrations of butanol that are detrimental to growth of the parental strains (prior to genetic modification for reduced production of AcrA and/or AcrB). Improved tolerance is to butanol compounds including 1-butanol, isobutanol, and 2-butanol. In addition, the present strains have improved tolerance to 2-butanone, which is also called methylethyl ketone (MEK). The amount of tolerance improvement will vary depending on the inhibiting chemical and its concentration, growth conditions and the specific genetically modified strain. For example, as shown in Example 7 herein, an acrA modified strain of E. coli showed improved growth over the parental strain that was about 5% improved growth in 0.8% 2-butanol, about 12% in 0.6% 2-butanol, about 3.5% in 0.6% isobutanol, and about 18% in 0.4% isobutanol. For example, as shown in Example 7 herein, an acrB modified strain of E. coli showed improved growth over the parental strain that was about 12% improved growth in 0.8% 2-butanol, about 24% in 0.6% 2-butanol, about 2.5% in 0.6% isobutanol, and about 20% in 0.4% isobutanol.

### Combined Genetic Modifications for Increased Tolerance

A separate genetic modification conferring butanol tolerance in bacterial cells is disclosed in commonly owned and co-pending U.S. Ser. No. 61/015,689 which is herein incorporated by reference. The additional modification is one that reduces accumulation of (p)ppGpp. Any genetic modification that reduces (p)ppGpp accumulation in an E. coli cell may be combined with a genetic modification that reduces AcrA and/or AcrB production to confer butanol tolerance. Specifically, modifications that reduce expression of spoT and/or re/A genes, or increase degradative activity relative to synthetic activity of SpoT, can reduce accumulation of (p)ppGpp. As summarized in Gentry and Cashel (Molec. Micro. 19:1373-1384 (1996)), the protein encoded by the spoT gene of E. coli (strain K12 coding region SEQ ID NO:55; protein SEQ ID NO:56) is an enzyme having both guanosine 3′5′-bis(diphosphate) 3′-pyrophosphohydrolase (ppGppase) and 3′,5′-bis(diphosphate synthetase (PSII) activities. In E. coli there is a closely related gene called re/A (strain K12 coding region SEQ ID NO:63; protein SEQ ID NO:64), which encodes an enzyme with 3′,5′-bis(diphosphate synthetase (PSI) activity. In E. coli, the RelA protein is associated with ribosomes and is activated by binding of uncharged tRNAs to the ribosomes. RelA activation and synthesis of (p)ppGpp results in decreased production of ribosomes, and stimulation of amino acid synthesis. The spoT gene product is responsible for synthesis of (p)ppGpp (Hernandez and Bremer, J. Biol. Chem. (1991) 266:5991-9) during carbon source starvation (Chaloner-Larsson andyamazaki Can. J. Biochem. (1978) 56:264-72; (Seyfzadeh and Keener, Proc. Natl. Acad.Sci. USA (1993) 90:11004-8) in E. coli.

As described for the E. coli acrA and acrB coding regions, coding regions for spoT and re/A from various strains of E. coli are readily recognized by one skilled in the art, having only few variations with sequence identities of at least about 96%, 97%, 98%, or 99% and are targets for modification in their host strains. For example, spoT coding regions and SpoT proteins, respectively, for different E. coli strains are as follows: E. coli o157:h7 SEQ ID NO:57 and SEQ ID NO:58; E. coli CFT073 SEQ ID NO:59 and SEQ ID NO:60; E. Coli UT189 SEQ ID NO:61 and SEQ ID NO:62. For example, re/A coding regions and RelA proteins, respectively, for different E. Coli strains are as follows: E. coli o157:h7 SEQ ID NO:65 and SEQ ID NO:66; E. coli CFT073 SEQ ID NO:67 and SEQ ID NO:68; E. coli UT189 SEQ ID NO:69 and SEQ ID NO:70.

In one embodiment of the present E. coli cell with combined genetic modification, both re/A and spoT genes are modified, causing reduced expression of both genes, to confer butanol tolerance. The spoT gene may be modified so that there is no expression, if expression of the re/A gene is reduced. Alternatively, with re/A unmodified, the expression of spoT may be lowered to provide increased tolerance. In addition, modification for reduced expression of re/A is sufficient to confer butanol tolerance under conditions where an aminoacyl-tRNA species is low and RelA production of (p)ppGpp would be high. Thus effects of the re/A mutation in limited aminoacyl-tRNA species conditions better exemplifies the impact on butanol tolerance of RelA-dependent (p)ppGpp synthesis. Elimination of spoT expression in a strain where re/A expression is reduced, (as demonstrated in Example 3 in commonly owned and co-owned and co-pending U.S. Ser. No. 61/015,689, which is herein incorporated herein by reference) confers butanol tolerance. Reduced expression of spoT in a strain where re/A expression is unmodified, (as demonstrated in Example 4 in commonly co-owned and co-pending U.S. Ser. No. 61/015,689, which is herein incorporated herein by reference), confers butanol tolerance.

Any genetic modification method known by one skilled in the art for reducing the presence of a functional enzyme may be used to alter spoT and/or re/A gene expression to reduce (p)ppGpp accumulation. Methods include, but are not limited to, deletion of the entire gene or a portion of the gene encoding SpoT or RelA, inserting a DNA fragment into the spoT or re/A gene so that the protein is not expressed or expressed at lower levels, introducing a mutation into the spoT or re/A coding region which adds a stop codon or frame shift such that a functional protein is not expressed, and introducing one or more mutations into the spoT or re/A coding region to alter amino acids so that a non-functional or a less enzymatically active protein is expressed. In addition, spoT or re/A expression may be blocked by expression of an antisense RNA or an interfering RNA, and constructs may be introduced that result in cosuppression. Moreover, a spoT or re/A gene may be synthesized whose expression is low because rare codons are substituted for plentiful ones, and this gene substituted for the endogenous corresponding spoT or re/A gene. Such a gene will produce the same polypeptide but at a lower rate. In addition, the synthesis or stability of the transcript may be lessened by mutation. Similarly the efficiency by which a protein is translated from mRNA may be modulated by mutation. All of these methods may be readily practiced by one skilled in the art making use of the known sequences encoding the E. coli SpoT or RelA enzyme. One skilled in the art may choose specific modification strategies to eliminate or lower the expression of the re/A or spoT gene as desired in the situations described above.

Alternatively, to reduce (p)ppGpp accumulation, a genetic modification may be made that increases the (p)ppGpp degradation activity present in an E. coli cell. The endogenous spoT gene may be modified to reduce the (p)ppGpp synthetic function of the encoded protein. A modified spoT gene encoding a protein with only degradative activity may be introduced. Regions of the SpoT protein that are responsible for the synthetic and degradative activities have been mapped (Gentry and Cashel Mol. Microbiol. (1996) 19:1373-1384). Domains of SpoT called RelA/SpoT, TGS, and HD were identified by Pfam (Pfam: clans, web tools and services: R. D. Finn, J. Mistry, B. Schuster-Bockler, S. Griffiths-Jones, V. Hollich, T. Lassmann, S. Moxon, M. Marshall, A. Khanna, R. Durbin, S. R. Eddy, E. L. L. Sonnhammer and A. Bateman, Nucleic Acids Research (2006) Database Issue 34:D247-D251). The RelA/SpoT and TGS domains of SpoT function in ppGpp synthesis while the HD domain is responsible for ppGpp hydrolysis. Gentry and Cashel showed that destruction of the HD domain eliminated the hydrolytic activity without loss of biosynthetic capacity while elimination of either of the other 2 domains resulted in loss of the synthetic capacity without loss of the hydrolytic activity. Thus the sequences encoding the RelA/SpoT and/or TGS domains in the endogenous spoT gene may be mutated to reduce (p)ppGpp synthetic activity. For example, in frame deletions eliminating the various domains can be readily synthesized in vitro and recombined into the chromosome by standard methods of allelic replacement. Examples of such deletions are readily found in the literature for both RelA (Fujita et al. Biosci. Biotechnol. Biochem. (2002) 66:1515-1523; Mechold et al J. Bacteriol. (2002) 84:2878-88) and SpoT (Battesti and Bouveret (2006) Molecular Microbiology 62:1048-10630). Furthermore, residual degradative capacity can be enhanced by increasing expression of the modified endogenous gene via chromosomal promoter replacements using methods such as described by Yuan et al (Metab. Eng. (2006) 8:79-90), and White et al. (Can. J. Microbiol. (2007) 53:56-62). Alternatively, a mutation affecting the function of either the RelA/SpoT domain or the TGS domain may be made in a spoT gene, and this gene introduced into an E. coli cell to increase (p)ppGpp degradation activity with no increase in synthesis.

DNA sequences surrounding the spoT or re/A coding sequence are useful in some modification procedures and are available for E. coli in the complete genome sequence of the K12 strain: GenBank Accession #U00096.2. In particular, DNA sequences surrounding the spoT or re/A coding sequence are useful for modification methods using homologous recombination. An example of this method is using spoT gene flanking sequences bounding a selectable marker gene to mediate homologous recombination whereby the marker gene replaces the spoT gene. Also partial spoT gene sequences and spoT flanking sequences bounding a selectable marker gene may be used to mediate homologous recombination whereby the marker gene replaces a portion of the spoT gene. In addition, the selectable marker may be bounded by site-specific recombination sites, so that following expression of the corresponding site-specific recombinase, the resistance gene is excised from the spoT gene without reactivating the latter. The site-specific recombination leaves behind a recombination site which disrupts expression of the SpoT enzyme. The homologous recombination vector may be constructed to also leave a deletion in the spoT gene following excision of the selectable marker, as is well known to one skilled in the art. Moreover, promoter replacement methods may be used to exchange the endogenous transcriptional control elements allowing another means to modulate expression (Yuan et al. ibid).

The spoT gene of E. coli is within a demonstrated operon. When part of an operon, expression of spoT or re/A may also be reduced by genetic modification of a coding region that is upstream of the spoT or re/A coding region in the operon. In the spoT-containing operon in E. coli, upstream of the spoT coding region are coding regions for gmk (guanosine monophosphate kinase) and rpoZ (DNA-directed RNA polymerase subunit omega). A modification of the gmk or rpoZ coding region which produces a polar effect will reduce or eliminate spoT expression. Polar mutations are typically nonsense, frameshift or insertion mutations. With these types of mutations, transcription may be truncated, translational coupling is prevented, and hence both interrupted and downstream genes are not expressed. This type of modification (described in Example 2 in commonly owned and of co-owned and co-pending U.S. Ser. No. 61/015,689, (which is herein incorporated herein by reference) where a transposon insertion in rpoZ affects spoT expression and butanol tolerance. In addition, in Examples 3 and 4 of commonly-owned and co-pending U.S. Ser. No. 61/015,689, (which is herein incorporated herein by reference), a polar modification in rpoZ was constructed resulting in butanol tolerance. In addition intergenic regions could be modified to prevent translational coupling when it is found.

Any genetic modification reducing SpoT and/or RelA production may be combined with any modification reducing AcrA and/or AcrB production. For example, Example 4 herein describes construction of a strain having an insertion in acrB and a polar mutation in rpoZ, which reduces expression of the spoT gene. As demonstrated in Example 5 herein, this acrB rpoZ double mutant had a higher growth yield than either single mutant Reduced response to (p)ppGpp

The effect of reducing accumulation of (p)ppGpp may also be obtained in the present strains by reducing responsiveness to (p)ppGpp. Any modification reducing AcrA and/or AcrB production may be combined with a modification reducing responsiveness to (p)ppGpp. Mutants with reduced response to (p)ppGpp were found in the RNA polymerase core subunit encoding genes and the RNA polymerase binding protein DksA (Potrykus and Cashel (2008) Ann. Rev. Microbiol. 62:35-51). Reduced expression of any of these proteins may be engineered to reduce the response to (p)ppGpp. In particular, reducing expression of DksA may be engineered in the present strains to confer increased tolerance to butanol and 2-butanone. Expression of the endogenous dksA gene in an E. coli host cell may be reduced using any genetic modification method such as described above for spoT or re/A. The dksA gene of E. coli is readily identified by one skilled in the art in publicly available databases.

Butanol or 2-butanone Biosynthetic Pathway

The present genetically modified E. coli strains with improved tolerance to butanol and 2-butanone are additionally genetically modified by the introduction of a biosynthetic pathway for the synthesis of butanol or 2-butanone. Alternatively, an E. coli strain having a biosynthetic pathway for the synthesis of butanol or 2-butanone may be genetically modified for reduced production of AcrA and/or acrB as described herein to confer butanol tolerance. The butanol biosynthetic pathway may be a 1-butanol, 2-butanol, or isobutanol biosynthetic pathway. In addition, a 2-butanone pathway may be present in the E. coli strain.

### 1-Butanol Biosynthetic Pathway

A biosynthetic pathway for the production of 1-butanol is described by Donaldson et al. in co-pending and commonly owned U.S. Patent Application Publication No. US20080182308A1, which is incorporated herein by reference. This biosynthetic pathway comprises the following substrate to product conversions:
a) acetyl-CoA to acetoacetyl-CoA, as catalyzed for example by acetyl-CoA acetyltransferase encoded by the genes given as SEQ ID NO:1 or 3;
b) acetoacetyl-CoA to 3-hydroxybutyryl-CoA, as catalyzed for example by 3-hydroxybutyryl-CoA dehydrogenase encoded by the gene given as SEQ ID NO:5;
c) 3-hydroxybutyryl-CoA to crotonyl-CoA, as catalyzed for example by crotonase encoded by the gene given as SEQ ID NO:7;
d) crotonyl-CoA to butyryl-CoA, as catalyzed for example by butyryl-CoA dehydrogenase encoded by the gene given as SEQ ID NO:9;
e) butyryl-CoA to butyraldehyde, as catalyzed for example by butyraldehyde dehydrogenase encoded by the gene given as SEQ ID NO:11; and
f) butyraldehyde to 1-butanol, as catalyzed for example by 1-butanol dehydrogenase encoded by the genes given as SEQ ID NO:13 or 15.

The pathway requires no ATP and generates NAD⁺ and/or NADP⁺, thus, it balances with the central, metabolic routes that generate acetyl-CoA.

### 2-Butanol and 2-Butanone Biosynthetic Pathway

Biosynthetic pathways for the production of 2-butanol and 2-butanone are described by Donaldson et al. in co-pending and commonly owned U.S. Patent Application Publication Nos. US20070259410A1 and US20070292927A1, which are incorporated herein by reference. One 2-butanol biosynthetic pathway comprises the following substrate to product conversions:
a) pyruvate to alpha-acetolactate, as catalyzed for example by acetolactate synthase encoded by the gene given as SEQ ID NO:19;
b) alpha-acetolactate to acetoin, as catalyzed for example by acetolactate decarboxylase encoded by the gene given as SEQ ID NO:17;
c) acetoin to 2,3-butanediol, as catalyzed for example by butanediol dehydrogenase encoded by the gene given as SEQ ID NO:21;
d) 2,3-butanediol to 2-butanone, catalyzed for example by butanediol dehydratase encoded by genes given as SEQ ID NOs:23, 25, and 27; and
e) 2-butanone to 2-butanol, as catalyzed for example by 2-butanol dehydrogenase encoded by the gene given as SEQ ID NO:29.
   Omitting the last step (e) of the above pathway provides a biosynthetic pathway for production of 2-butanone, also known as methyl ethyl ketone (MEK).

### Isobutanol Biosynthetic Pathway

Biosynthetic pathways for the production of isobutanol are described by Maggio-Hall et al. in copending and commonly owned U.S. patent application Ser. No. 11/586,315, published as US20070092957 A1, which is incorporated herein by reference. One isobutanol biosynthetic pathway comprises the following substrate to product conversions:
a) pyruvate to acetolactate, as catalyzed for example by acetolactate synthase encoded by the gene given as SEQ ID NO:19;
b) acetolactate to 2,3-dihydroxyisovalerate, as catalyzed for example by acetohydroxy acid isomeroreductase encoded by the gene given as SEQ ID NO:31;
c) 2,3-dihydroxyisovalerate to α-ketoisovalerate, as catalyzed for example by acetohydroxy acid dehydratase encoded by the gene given as SEQ ID NO:33;
d) α-ketoisovalerate to isobutyraldehyde, as catalyzed for example by a branched-chain keto acid decarboxylase encoded by the gene given as SEQ ID NO:35; and
e) isobutyraldehyde to isobutanol, as catalyzed for example by a branched-chain alcohol dehydrogenase encoded by the gene given as SEQ ID NO:37.
   Construction of E coli Strains for Butanol or Butanone Production

Any E coli strain that is genetically modified for butanol tolerance as described herein is additionally genetically modified (before or after modification to tolerance) to incorporate a butanol or 2-butanone biosynthetic pathway by methods well known to one skilled in the art. Genes encoding the enzyme activities described above, or homologs that may be identified and obtained by commonly used methods well known to one skilled in the art, are introduced into an E coli host. Representative coding and amino acid sequences for pathway enzymes that may be used are given in Tables 1, 2, and 3, with SEQ ID NOs:1-38. Methods described in co-pending and commonly owned U.S. Patent Application Publication Nos. US20080182308A1, US20070259410A1, US20070292927A1, and US20070092957 A1 may be used.

Vectors or plasmids useful for the transformation of E coli cells are common and commercially available from companies such as EPICENTRE (Madison, Wis.), Invitrogen Corp. (Carlsbad, Calif.), Stratagene (La Jolla, Calif.), and New England Biolabs, Inc. (Beverly, Mass.). Typically, the vector contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the E coli host cell are numerous and familiar to those skilled in the art. Virtually any promoter capable of driving these genetic elements is suitable for the present invention including, but not limited to, lac, ara, tet, trp, λP_{L}, λP_{R}, T7, tac, and trc.

Termination control regions may also be derived from various genes native to the preferred hosts. Optionally, a termination site may be unnecessary, however, it is most preferred if included.

Certain vectors are capable of replicating in a broad range of host bacteria including E coli and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. Additionally, in vitro transposomes are available to create random mutations in the E coli genome from commercial sources such as EPICENTRE (Madison, Wis.).

Fermentation of Butanol Tolerant E coli for Butanol or 2-butanone Production

The present strains with reduced AcrA and/or AcrB production and having a butanol or 2-butanone biosynthesis pathway may be used for fermentation production of butanol or 2-butanone. Fermentation media for the production of butanol or butanone must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Sucrose may be obtained from feedstocks such as sugar cane, sugar beets, cassaya, and sweet sorghum. Glucose and dextrose may be obtained through saccharification of starch based feedstocks including grains such as corn, wheat, rye, barley, and oats.

In addition, fermentable sugars may be obtained from cellulosic and lignocellulosic biomass through processes of pretreatment and saccharification, as described, for example, in commonly owned and co-pending US patent application publication US20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

Although it is contemplated that all of the above mentioned carbon substrates and mixtures thereof are suitable in the present invention, preferred carbon substrates are glucose, fructose, and sucrose.

In addition to an appropriate carbon source, fermentation media must contain suitable minerals, salts, cofactors, buffers and other components, known to those skilled in the art, suitable for the growth of the cultures and promotion of the enzymatic pathway necessary for butanol or butanone production.

Typically cells are grown at a temperature in the range of about 25° C. to about 40° C. in an appropriate medium. Suitable growth media are common commercially prepared media such as Bacto Lactobacilli MRS broth or Agar (Difco), Luria Bertani (LB) broth, Sabouraud Dextrose (SD) broth or Yeast Medium (YM) broth. Other defined or synthetic growth media may also be used, and the appropriate medium for growth of the particular E. coli strain will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between pH 5.0 to pH 9.0, where pH 6.0 to pH 8.0 is preferred as the initial condition.

Fermentations may be performed under aerobic or anaerobic conditions.

Butanol or butanone may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol or butanone may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of butanol or butanone may be practiced using either batch, fed-batch or continuous processes and that any known mode of fermentation would be suitable. Additionally, it is contemplated that cells may be immobilized on a substrate as whole cell catalysts and subjected to fermentation conditions for butanol or butanone production.

Any set of conditions described above, and additionally variations in these conditions that are well known to one skilled in the art, are suitable conditions for production of butanol or 2-butanone by the present acrA and/or acrB modified recombinant E. coli strains.

Methods for Butanol and 2-Butanone Isolation from the Fermentation Medium

Bioproduced butanol may be isolated from the fermentation medium using methods known in the art for ABE fermentations (see for example, Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the butanol may be isolated from the fermentation medium using methods such as distillation, azeotropic distillation, liquid-liquid extraction, adsorption, gas stripping, membrane evaporation, or pervaporation. These same methods may be adapted to isolate bioproduced 2-butanone from the fermentation medium.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “sec’ means second(s), “μl” means microliter(s), “ml” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “rpm” means revolutions per minute, “w/v” means weight/volume, “OD” means optical density, and “OD600” means optical density measured at a wavelength of 600 nm.

### General Methods:

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987. Additional methods used in the Examples are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, (1990), and A Short Course in Bacterial Genetics (Miller, Cold Spring Harbor Laboratory (1992)).

These references include descriptions of the media and buffers used including TE, M9, MacConkey and LB.

All reagents, restriction enzymes and materials used for the growth and maintenance of bacterial cells were obtained from Promega Corporation (Madison, Wis.), Teknova Corporation (Hollister, Calif.), MediaTech, Inc. (Herndon, Va.), Applied Systems (Foster City, Calif.), Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Freezing Medium

The following medium was used to store cells in microtitre plates. Stock solutions (autoclaved each solution after making):
0.68 M Ammonium Sulfate (NH₄)₂SO₄: 44.95 g, brought to 500 mL with dlH₂O
0.04 M Magnesium Sulfate MgSO₄: 2.4 g, brought g to 500 mL with dlH₂O
0.17 M Sodium Citrate: 25 g, brought g to 500 mL with dlH₂O
1.32 M KH₂PO₄: 17.99 g, brought to 100 mL with dlH₂O
3.6 M K₂HPO₄: 62.7 g, brought to 100 mL with dlH₂O
   To make 10× freezing medium, 138.6 g glycerol was weighed into a tared 250 mL plastic beaker. 25 mL of each of the above five stock solutions were added with stirring mediated with a magnetic stirrer and a stir plate until thoroughly mixed. Distilled water was added until a final volume of 250 mL was achieved. The solution was filtered through a 0.2 micron sterile filter. To use, a 1 volume of 10× freezing medium was added to 9 volumes of LB. The final concentrations are: 36 mM K₂HPO₄, 13.2 mM KH₂PO₄, 1.7 mM Sodium Citrate, 0.4 mM MgSO₄, 6.8 mM (NH₄)₂SO₄, 4.4% v/v glycerol in LB. Sterile flat-bottomed clear polystyrene 96-well plates (Corning Costar #3370, pre-bar-coded) were used for storing libraries of mutants in freezing medium in a −80° C. freezer.

### Agar Plates

LB agar media supplemented with butanol was prepared fresh one day before innoculating at an appropriate volume and cooled for 2 hours in a 50° C. water bath. LB agar plates supplemented with butanol were prepared by dispensing 67 mls of melted agar, using a peristaltic pump and sterile Nalgene tubing, into sterile Omni trays with lids (Nunc mfg no. 242811). The 1-butanol (Sigma Aldrich, Part No. B7906-500 ml) was added and mixed by vigorous swirling immediately before dispensing the agar to minimize evaporation of the butanol. The plates were allowed to cool and set for approximately an hour before they were stored overnight in closed anaerobic chambers at room temperature in the chemical/biological hood. The next morning, the chambers harboring the plates were opened and allowed to air dry for approximately 1 hour before using.

Methods for Determining Isobutanol, 1-butanol, 2-butanol, and 2-butanone Concentration in Culture Media

The concentration of isobutanol in the culture media can be determined by a number of methods known in the art. For example, a specific high performance liquid chromatography (HPLC) method utilized a Shodex SH-1011 column with a Shodex SH-G guard column, both purchased from Waters Corporation (Milford, Mass.), with refractive index (RI) detection. Chromatographic separation was achieved using 0.01 M H₂SO₄ as the mobile phase with a flow rate of 0.5 mL/min and a column temperature of 50° C. Isobutanol had a retention time of 46.6 min under the conditions used. 1-Butanol had a retention time of 52.8 min under the conditions used. Under the conditions used, 2-butanone and 2-butanol had retention times of 39.5 and 44.3 min, respectively.

Alternatively, gas chromatography (GC) methods are available. For example, a specific GC method utilized an HP-INNOWax column (30 m×0.53 mm id, 1 μm film thickness, Agilent Technologies, Wilmington, Del.), with a flame ionization detector (FID). The carrier gas was helium at a flow rate of 4.5 mL/min, measured at 150° C. with constant head pressure; injector split was 1:25 at 200° C.; oven temperature was 45° C. for 1 min, 45 to 220° C. at 10° C./min, and 220° C. for 5 min; and FID detection was employed at 240° C. with 26 mL/min helium makeup gas. The retention time of isobutanol was 4.5 min. The retention time of 1-butanol was 5.4 min. The retention times of 2-butanone and 2-butanol were 3.61 and 5.03 min, respectively.

### Example 1

### Generation of Knockout Library and Screening to Identify 1-Butanol Phenotypes

E. coli strain EC100 (Epicentre; Madison, Wis.], whose genotype is F-mcrA Δ (mrr-hsdRMS-mcrBC) φ80dlac [symbol] M15 ΔlacX74 recA1 relA1 endA1 araD139 Δ(ara, leu)7697 galU galK λ-rpsL nupG, was transposome mutagenized. This was performed according to the vendor's (Epicentre; Madison, Wis.) protocol, using purchased electro-competent cells as the recipient in the genetic cross with the EZ-Tn5™<KAN-2>Tnp Transposome™. 1 μl of the EZ-Tn5<KAN-2>Tnp Transposome was electroporated into EC100 cells. Immediately after electroporation, SOC medium was added to a final volume of 1 ml and the mixture was gently agitated before transfer to a tube that was incubated at 37° C. with shaking for 1 hr. The genetic cross yielded a titer ranging from 4 to 7×10⁴ kanamycin-resistant colony-forming units per ml of electroporated cells.

100 μl aliquots of undiluted cells and dilutions were separately plated on LB medium containing 50 μg/ml kanamycin to yield about 500 colonies per plate, that could be picked and stored. This process utilized a robotic AutoGenesys Colony Picker to select individual colonies from 22 cm² LB kanamycin (50 μg/mL) agar plates. The colony picker used a CCD camera image with select parameters to discriminate colonies for picking based on size, roundness, and proximity to other colonies. For size, the parameters were 0.5 mm to 1.8 mm for small cells, 1.8 to 3.0 mm for large cells. Roundness determinations were made from 1.30 mm ellipticity with a 1.50 mm variance for small cells, and 1.50 mm ellipticity with a 1.50 mm variance for large cells. The cells also had to be 1.3 mm or 500 pixels apart from neighboring cells. The individual, well-separated colonies were imaged and picked to media-containing microtiter wells. The colonies were picked into 92 of the 96 wells of archive microtiter plates containing 150 μl per well of freezing medium supplemented with 50 μg/ml kanamycin (see General Methods). Four wells were left blank and served as negative controls. The archive plates were lidded and placed in a humidified static incubator at 37° C. for overnight incubation. The plates were then placed in −80° C. storage for future use. The record of archive plate barcode IDS were transferred from the colony picker to the Blaze Systems Laboratory Information System (LIMS). A total of 11,886 colonies were picked to the microtiter wells. This library was expected to have a 90% probability of containing a mutation inactivating any non-essential gene, which would be a mutation in 3600 of a possible 4000 ORFs.

To determine inhibitory 1-butanol concentrations, strain EC100 was grown overnight in LB medium and aliquots of various dilutions were plated on solidified LB medium appended with concentrations of 1-butanol up to 1% at 0.1% integrals. Plates were incubated in a closed chamber at 37° C. for 1 day. The number of colonies arising and their sizes were scored. Colonies were progressively smaller starting at 0.2% 1-butanol, with only pinpoint colonies seen at 0.6%. No change in titer was seen in the range of 0 to 0.6%. No colony formation after overnight incubation was observed at concentrations ≧0.7% (w/v). Butanol concentrations of 0.4% and 0.6% were chosen to screen for tolerance.

For screening of the transposon library, archive plates were removed from −80° C. storage and allowed to thaw at room temperature for an hour. Using a 96-pin HDRT (high density replication tool) on a Biomek 2000 robot, an archive plate was sampled multiple times with inocula printed on multiple agar plates. The final agar plate was an LB plate used as a quality control for verifying instrument and experimental conditions. The Biomek printing method employed a pin decontamination step at both the beginning and the end of each run. The pins were dipped first into 10% bleach solution (10 sec.), followed by water and 70% ethanol dips (10 sec. each). The pins were then dried over a room temperature fan (25 sec.). The archive plates were returned to the −80° C. freezer.

The control printed agar plates were lidded, put into plastic bags, and placed in a 37° C. incubator. Printed plates containing 1-butanol were handled in a chemical fume hood where they were placed in sealed portable anaerobic chambers: 7.0 liter AnaeroPack Rectangular Jars (Remel Inc.; Lenexa, Kans.).

Incubation at 20° C. or 37° C. was performed for 2 days; scoring was done on both days. Scoring of 1-butanol-containing plates was performed in a chemical hood. A visual screen identified 23 variants which grew slightly better than their neighbors on the butanol containing plates.

### Example 2

### Mapping of Transposon Insertions in 1-Butanol Tolerant Strains

In order to link 1-butanol phenotypic alterations with a gene/protein/function, the transposon insertion positions were determined by sequencing. Genomic DNA was prepared from the identified 1-butanol tolerant lines using a GenomiPhi™ DNA Amplification kit (GE/Amersham Biosciences; Piscataway, N.J.) which utilizes Phi29 DNA polymerase and random hexamers to amplify the entire chromosome, following the manufacturer's protocol. A portion of a colony from a culture plate was diluted in 100 μl of water, and 1-2 μl of this sample was then added to the lysis reagent and heated for 3 minutes at 95° C. and cooled to 4° C. Next the polymerase was added and the amplification proceeded overnight at 30° C. The final step was enzyme inactivation for 10 minutes at 65° C. and cooling to 4° C.

The resulting genomic DNA was sequenced using the following primers that read outward from each end of the transposon:

| | |
|---|---|
| Kan2cb-Fwd: | |
| CTGGTCCACCTACAACAAAGCTC TCATC | SEQ ID NO:72 |
| Kan2cb-Rev: | |
| CTTGTGCAATGTAACATCAGAGATTTTGAGACAC. | SEQ ID NO:73 |

From each 20 μl GenomiPhi™ amplified sample, 8 μl was removed and added to 16 μl of BigDye v3.1 Sequencing reagent (PN #4337457; Applied Biosystems; Foster City, Calif.), 3 μl of 10 μM primer (SEQ ID NO:1 or 2), 1 μl Thermofidelase (Fidelity Systems; Gaithersburg, Md.) and 12 μl Molecular Biology Grade water (Mediatech, Inc.; Herndon, Va.). The sequencing reactions were then thermal cycled as follows; 3 minutes at 96° C. followed by 200 cycles of (95° C. 30 sec+55° C. 20 sec+60° C. 2 min), then stored at 4° C. The unincorporated ddNTPs were removed prior to sequencing using Edge Biosystems (Gaithersburg, Md.) clean-up plates. For each sequencing reaction the total 40 μl was pipetted into one well of a pre-spun 96-well clean up plate. The plate was then spun for 5 min at 5,000×g in a Sorvall RT-7 refrigerated centrifuge. The cleaned up reactions were then placed directly onto an Applied Biosystems 3700 DNA sequencer and sequenced with automatic base-calling.

The sequences that were obtained were aligned with the E. coli K12 genome using BLAST (2.2.9, Basic Local Alignment Search Tool). The output was a string of matched nucleotides within the E. coli genome designated by nucleotide number, which then was used to identify open reading frames into which each transposon was inserted, using the EcoCyc database (SRI International; Menlo Park, Calif.)

In two separate strains, the transposon insertion was in the acrB coding region. These strains were named DPD1852 and DPD1858.

### Example 3

### 1-Butanol Tolerant Mutant Phenotype in Liquid Cultures

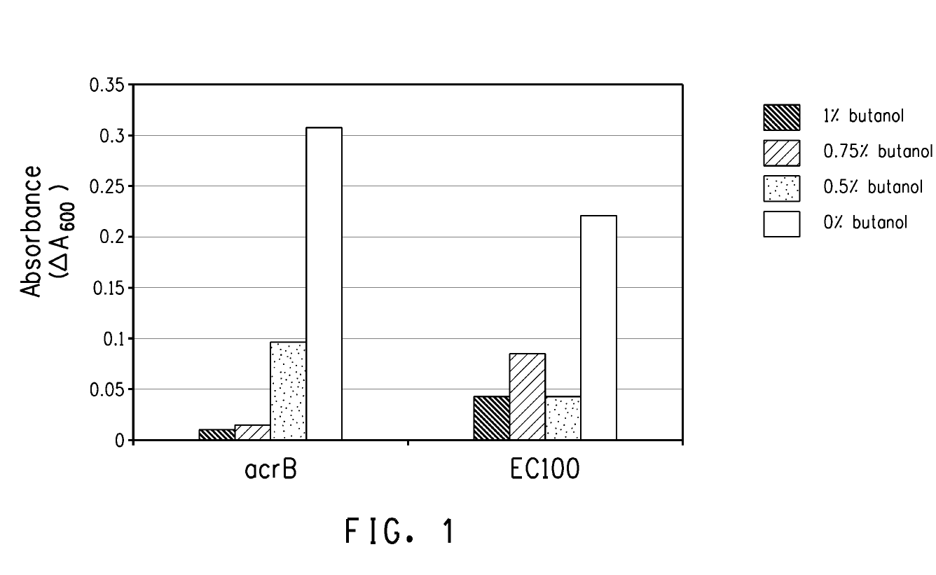

An acrB transposition mutant strain isolated in the above examples (DPD1852) and the EC100 parental line were cultured overnight with shaking at 37° C. in LB before 1:100 dilution in fresh LB. After al hr incubation, the culture was split into 1 ml aliquots (microfuge tubes) and 1-butanol was added to 0, 0.5%, 0.75% or 1% (w/v). After a further 2 hr incubation at 37° C. with shaking, 200 μl samples were transferred to a microtiter plate and optical density at A₆₀₀ recorded. The microtiter plate was moved to a platform shaker that was located within a plastic box that is in a 37° C. incubator. Optical density was subsequently recorded at 4 hour and the results are shown in FIG. 1 as the difference between the 4 and 2 hr time points.

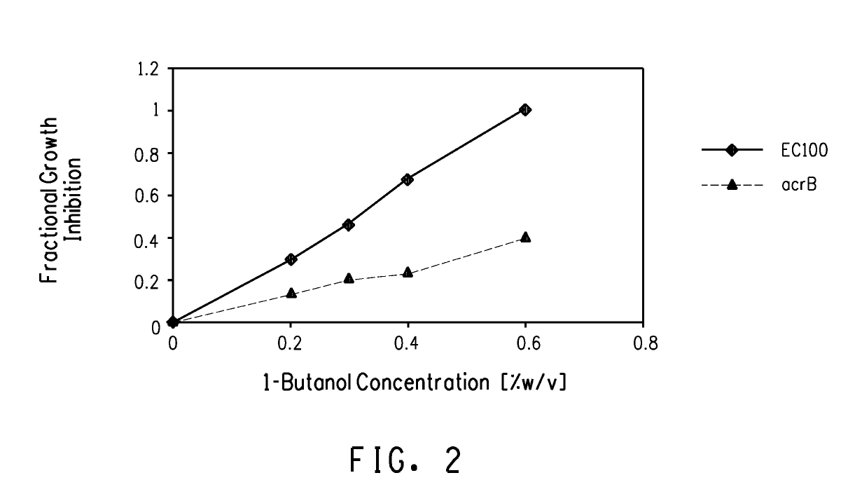

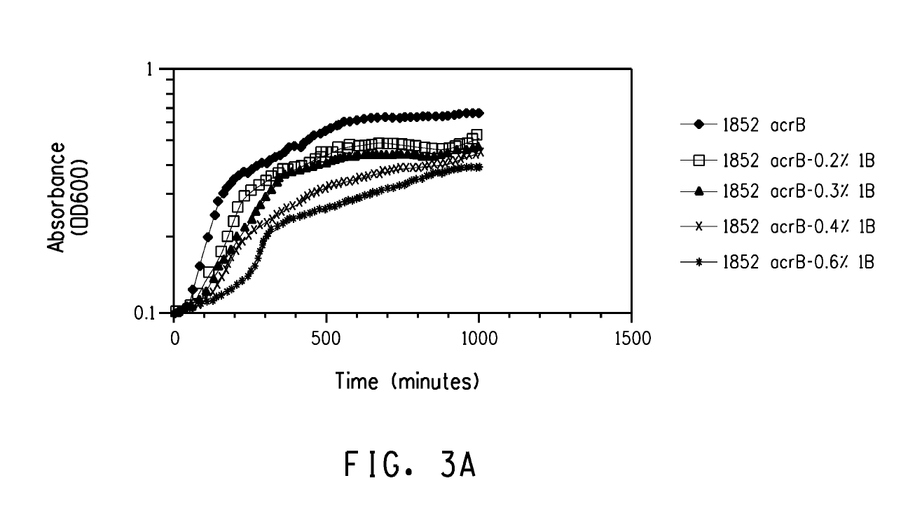

Kinetic growth studies were performed for the acrB transposition mutant strain and the control (EC100) using the Bioscreen C Automated Microbial Growth Curve Analyis System (Oy Growth Curves Ab Ltd., Helsinki, Finland), which is an automated 96 well plate system, that monitors growth of many cultures simultaneously, each in a volume of 150 μl. Overnight triplicate cultures of each strain were grown and diluted (1:10) into either LB or LB freshly supplemented with 0.2%, 0.3%. 0.4% or 0.6% 1-butanol (w/v). The growth of each culture was followed for approximately 18 hours. The triplicates were averaged and plotted in FIG. 2 as the final 18 hour time point, normalized to EC100, and given as the percent growth inhibition relative to the no butanol control for each strain.

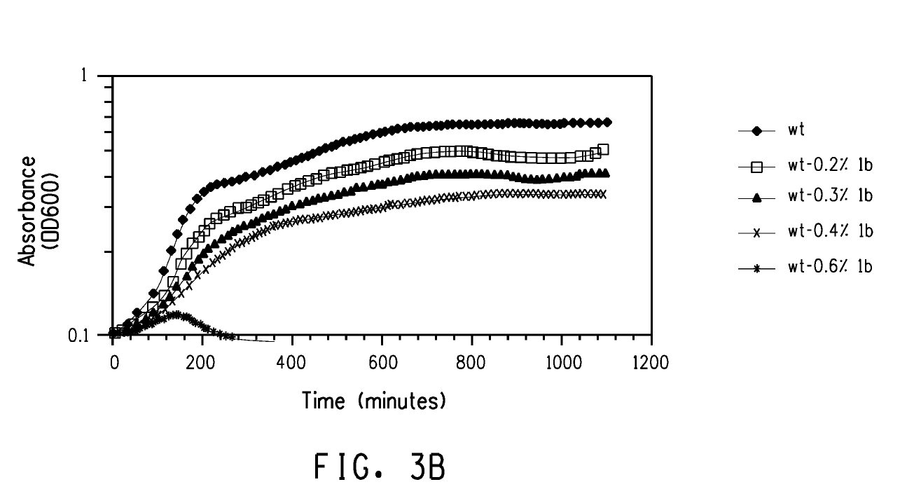

An additional kinetic growth study was performed as described above. The data is shown in FIG. 3 plotted as OD600 over time for the acrB transposition mutant strain (A) and wild type (B, EC100). The acrB mutant was more tolerant to all of the concentrations of 1-butanol tested than the wild type strain in terms of growth rate.

### Example 4

### Construction of Double Mutant to Increase Tolerance

A strain of E. coli was constructed to contain mutations that reduce expression of both the acrB gene and the spoT gene. A strain of E. coli K12 having an insertion in the acrB coding region was obtained from the Keio knockout collection (Baba et al. (2006) Mol. Syst. Biol. 2:2006.0008). This is a collection of lines, each with a kanamycin marker insertion in an identified location, made in the BW25113 strain (Coli Genetic Stock Center #: 7636; Datsenko, and Wanner (2000) Proc. Natl. Acad. Sci. USA 97:6640-6645). The acrB knockout line, called JW0451, served as the starting strain for the construction. The Keio collection also contains a strain having an insertion in the rpoZ coding region (called JW3624), that was used in the construction.

Reduced expression of spoT is described and shown in commonly-owned and co-pending U.S. Ser. No. 61/015,689, (which is herein incorporated herein by reference) to increase tolerance to butanol. In this Example a combination of reduced spoT and acrB expression is assessed. To reduce expression of spoT in the BW25113 strain, a polar mutation was made in the rpoZ coding region, which is upstream of the spoT coding region in the same operon. A spoT knockout was not constructed since this mutation combined with the relA+ phenotype of the BW25113 cell line is known to be lethal (Xiao et al. (1991) J. Biol. Chem. 266(9):5980-90). The constructed mutation (an insertion-deletion or indel) in rpoZ reduces expression of the spoT coding region since spoT is downstream of rpoZ in the operon containing these two coding regions

The Keio acrB mutant line (JW0451) has a kanamycin resistance marker gene flanked by FRT sites replacing most of the acrB coding region (described in Baba et al., supra). To construct the double mutant strain, first the Flp recombinase system was utilized to excise the kanamycin resistance marker flanked by FRT (FLP recognition) sites in the acrB gene, then an rpoz;;kan allele was introduced into the genome via homologous recombination.

The acrB mutant line was transformed with plasmid pCP20 (Cherepanov and Wackernagel (1995) Gene 158: 9-14) selecting for the plasmid encoded ampicillin resistance that has the Flp recombinase under lambda cl857 control in a replicon that cannot be maintained at high temperature as described in Baba et al. (supra). Transformants were grown on LB at high temperature (42° C.) to induce Flp expression by inactivating the Lambda repressor, and to cause plasmid loss. Clones were screened for kanamycin sensitivity which indicated that the kanamycin marker had been excised by the Flp recombinase. In addition, loss of a plasmid encode drug resistance marker indicated that the plasmid had been cured. Following excision, a single FRT recombination site remains in the acrB coding region, which does not disrupt expression of downstream genes. Most of the acrB coding sequence was deleted in the original Keio mutant line construction, so that acrB is not expressed. Kanamycin sensitive clones were screened for ampicillin sensitivity, which indicated loss of the pCP20 plasmid. The resulting acrB deletion line with both the pCP20 plasmid and the kanamycin resistance marker removed was called DPD1876.

To make the acrB and rpoZ double mutant, the rpoZ::kan allele of the Keio rpoZ mutant line (JW3624), which has a kanamycin resistance marker gene insertion in rpoZ, was transferred into DPD1876 as follows. A P1 lysogen of JW3624 was prepared (according to Miller, 1972) by first growing the cells to mid-logarithmic phase in LB at 37° C. and adding CaCl₂ (5 mM final concentration) before a 10 minute incubation on ice. A P1clr100CM phage (Miller, 1972, supra) was added at various multiplicities (0.5 μl or 5 μl) to 100 μl of calcium chloride-treated cells and absorbed at 30° C. for 30 minutes. The contents of the genetic cross were plated onto LB plates supplemented with chloramphenicol (25 μg/ml). Then single colonies were tested for lysogeny by monitoring temperature sensitivity by incubating on LB plates at 30° C. and 42° C. while also checking chloramphenicol and kanamycin resistance markers. The lysogen was grown at 30° C. in LB medium containing 10 mM MgSO₄ with shaking at 300 rpm for approximately 2 hours until an OD600 of approximately 0.1 was reached, and then shifted to 42° C. for 35 minutes to induce a phage lytic cycle due to inactivation of the thermo-labile repressor encoded by the clr100 allele of the P1 phage. The culture was then transferred to 39° C. for an additional 60 minutes to allow lysis to occur. The culture was centrifuged at top speed at 4° C. in a benchtop centrifuge, followed by addition of 0.1 ml of chloroform to the supernatant to kill any remaining cells, producing a transducing lysate.

This transducing lysate was mixed with DPD1876 cells for homologous recombination mediated gene replacement following standard protocols for generalized transduction of E. coli (Miller, supra). This was achieved by growing the DPD1876 strain in LB overnight, resuspending the culture in MC buffer (0.1 MgSO₄, 5 mM CaCl₂), and incubating at 37° C. for 15 minutes. Various dilutions of the transducing phage lysate were mixed with the treated recipient cells, which were then incubated at 30° C. for 30 minutes statically. The cells were plated onto LB plates containing kanamycin and incubated at 30° C. for 1 to 2 days. The transductants were single colony purified two times on LB plates containing kanamycin, then tested for absence of lysogeny (growth at 42° C.) and the desired constellation of drug phenotypes (kanamycin resistance and chloramphenicol sensitivity). The resulting double mutant strain with acrB rpoZ::kan, was called DPD1899. The polar kanamycin resistance cassette was maintained within rpoZ to minimize the downstream spoT expression.

### Example 5

### Growth Analysis of Constructed Double Mutant Line

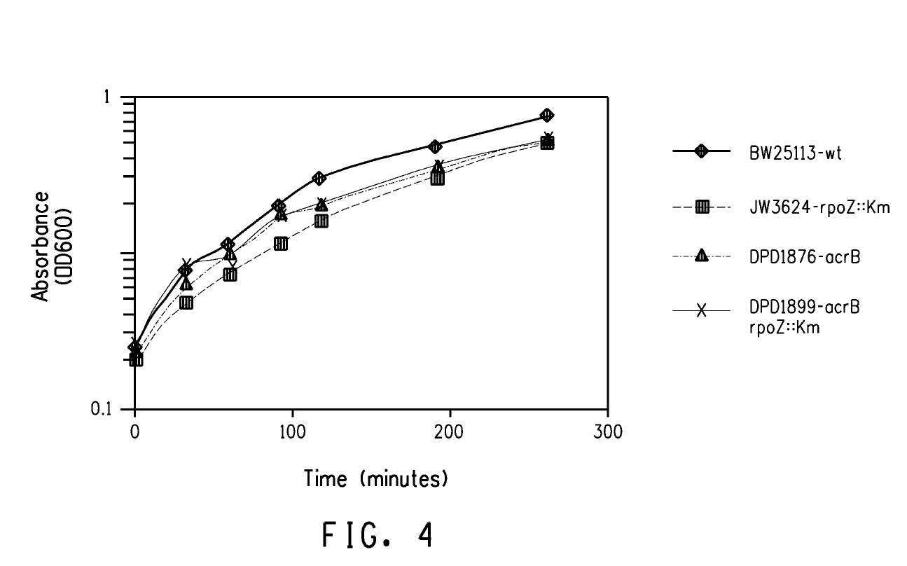

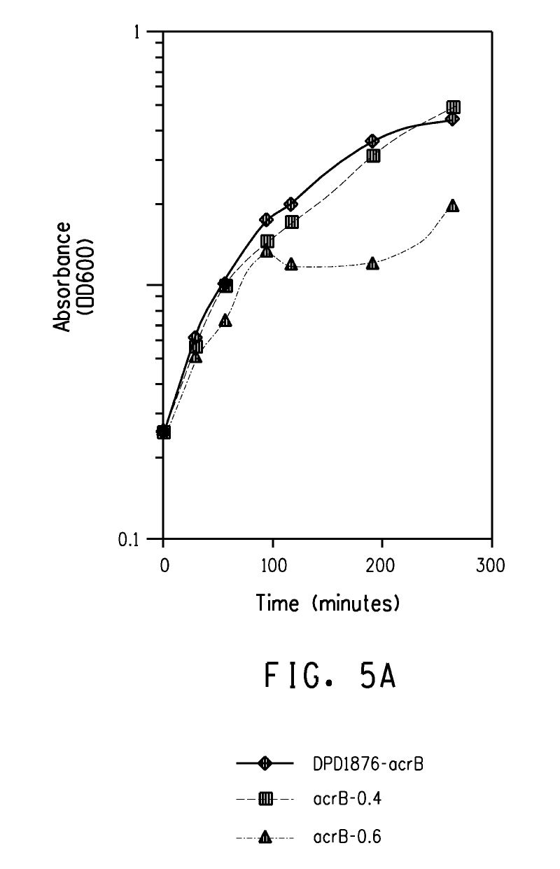

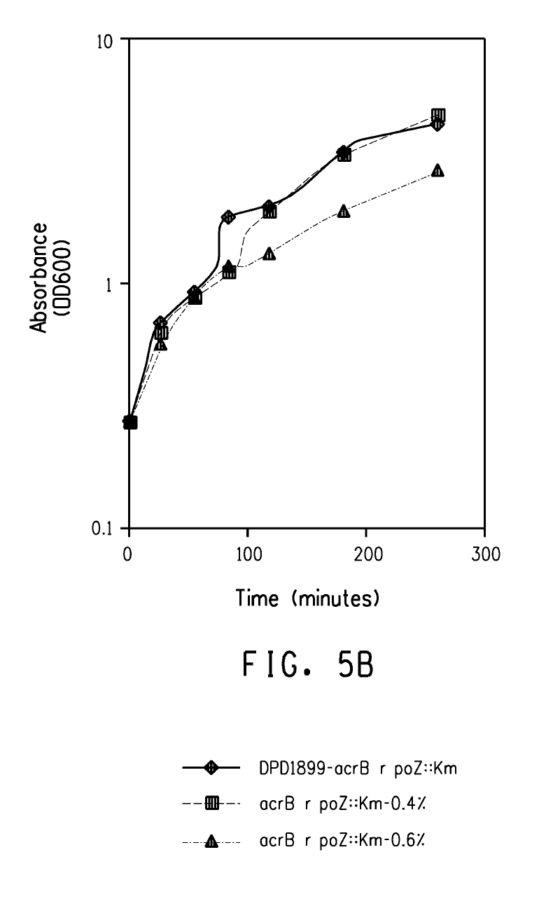

Shake flask experiments were performed on the acrB rpoZ::kan constructed double mutant line DPD1899, as well as the acrB deletion line DPD1876, the rpoZ::kan line JW3624, and the wild type control BW25113 line. Cultures were grown in LB medium containing 0%, 0.4% or 0.6% 1-butanol in shake flasks. The experiments were performed by inoculating 100 ml of medium in a 250 ml plastic flask with 2 ml of an overnight culture grown from a single colony grown at 37° C. and incubating with shaking for approximately two doubling times (1 hour), to an OD600 between 0.2 and 0.3. Each culture was split into five 25 ml cultures in plastic screw top 125 ml flasks and the cultures were maintained at 37° C. in a shaking water bath at 200 rpm. The OD600 was monitored at 0, 30, 90, 120 190, and 260 minutes. The growth data in the absence of 1-butanol is shown in FIG. 4. The growth data in the presence of 0.4% or 0.6% 1-butanol for DPD1876 and DPD1899 are shown in FIGS. 5 A and B, respectively.

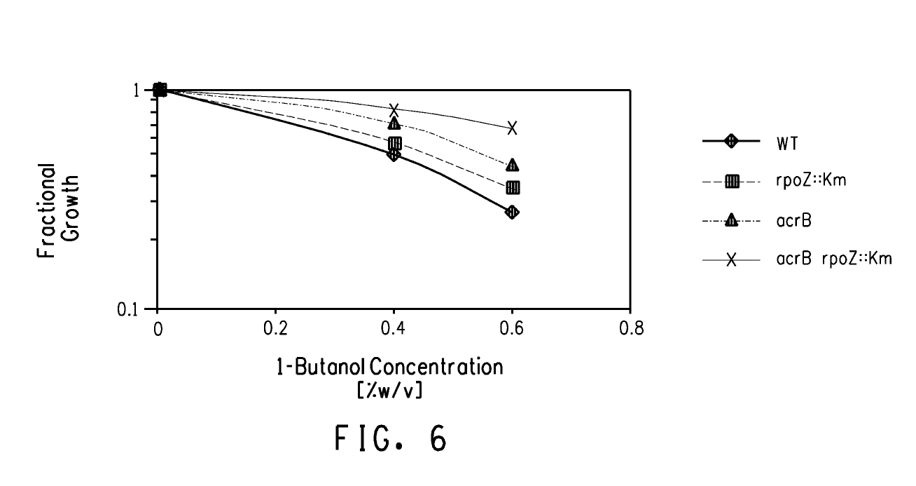

In the cultures above, a final time point was taken at 18 hr and used to calculate growth yield as a function of 1-butanol challenge. For each line grown in 0, 0.4% or 0.6% 1-butanol, the final 18 hour time point was divided by the no 1-butanol 18 hr time point. The results are shown in FIG. 6 as fractional growth. The results showed that the wild type cells were the most sensitive to growth inhibition in the presence of 0.4 and 0.6% 1-butanol. The rpoZ and acrB mutants had higher growth yields than wild type, and the acrB rpoZ double mutant had an even higher growth yield.

### Example 6

### Tolerance of acrB Mutant to Different Butanols

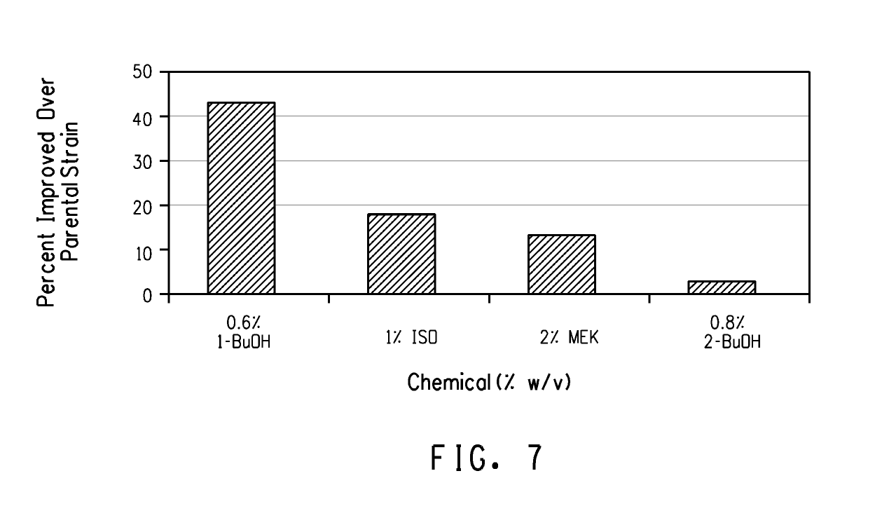

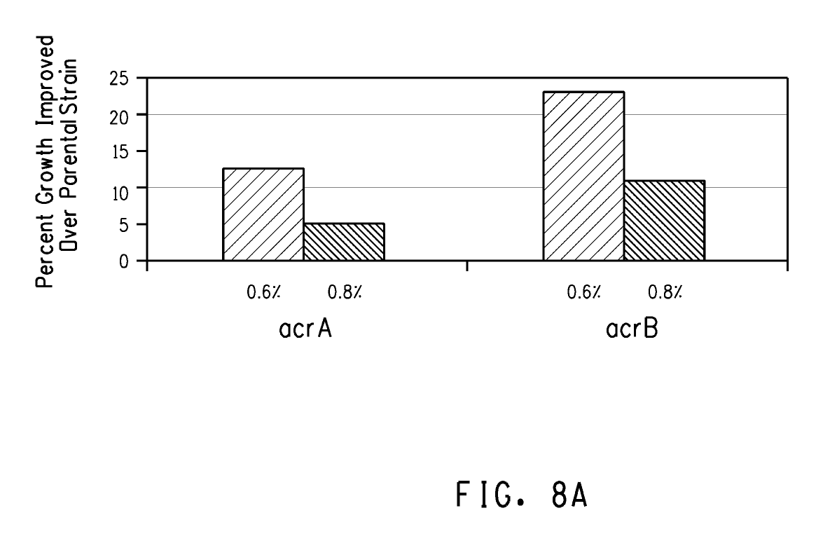

Growth of the acrB transposon mutant line, DPD1852 (in Example 2), and the parental strain EC100 were compared in the presence of different butanols. Cultures were grown in LB medium at 37° C. to mid-logarithmic phase and then 200 μl was put into microtiter wells of a Bioscreen device that monitors optical density as a function of time. Triplicate wells contained a culture challenged with a specified concentration of a compound. For 2- and isobutanol the concentrations tested were 0, 0.8, 1.2, 1.4, and 1.6% weight percent. For 1-butanol the concentrations were 0, 0.2, 0.3, 0.4, and 0.6% w/v. For 2-butanone (MEK, methylethylketone) the concentrations were 0, 2.5, 3, 3.5 and 4% w/v. Triplicate averaged culture density as a function of time for each condition was determined and the growth rates and final (18 hr) growth yields showed significant improvements due to the acrB mutation in comparison to the parental line. The percent growth improvement over the 18 hr time of exposure at indicated concentrations of various chemicals as graphed in FIG. 7 shows that the acrB mutation improved the tolerance to 1-butanol, isobutanol, 2-butanol and MEK.

### Example 7

### Comparative Butanol Tolerance of acrA and acrB Mutant Lines

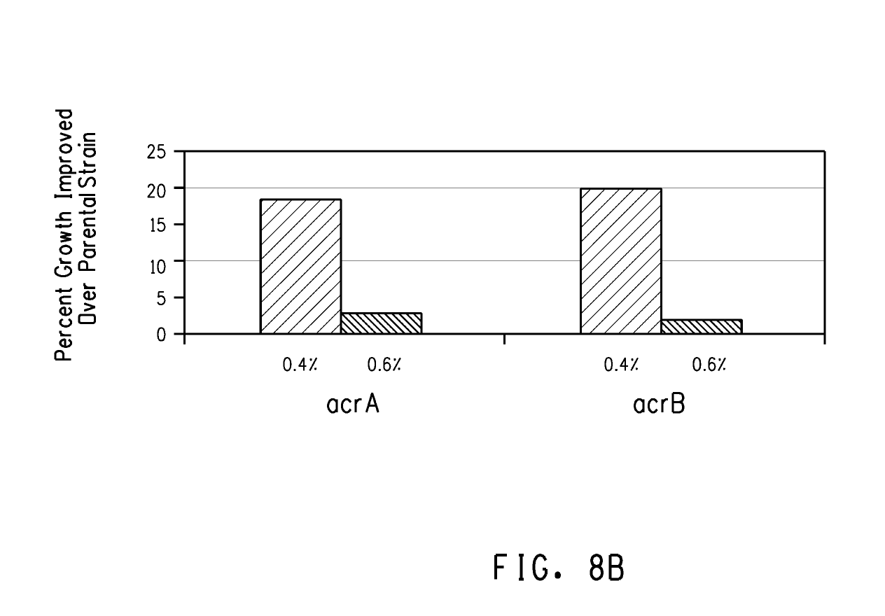

Strains of E. coli K12 EC100 having insertions in either the acrA or acrB coding regions, JW0452 and JW0451 respectively, were obtained from the Keio knockout collection described in Example 4. Growth of these strains was compared in the presence of 2-butanol and isobutanol in relation to the parental strain EC100. Overnight cultures were inoculated with a fresh colony and grown in LB at 37° C. with shaking. The next day the culture was diluted 1:100 into 100 ml of fresh LB in a 1 liter flask and grown for approximately 2 hours. The culture was split into 20 ml aliquots in 125 ml plastic screw top flasks. One culture remained unaltered serving as the no add control, and various concentrations of either 2-butanol or isobutanol were added to the remaining flasks. Absorbance (OD600) was monitored over time. Fractional growth yields were determined after 3 hr of exposure and percent improvement was calculated by subtracting the mutant fractional growth from that of wild the parental strain and multiplying by 100. Results given in FIG. 8 show that the acrA and acrB mutants are more tolerant to 2-butanol (FIG. 8A) and isobutanol (FIG. 8B) than their parent strain.

In addition, a to/C transposon insertion mutant of the Keio collection, JW5503, was also assayed. Growth of this strain was found to be indistinguishable from the parental strain in terms of its responses to 2-butanol and isobutanol.

### Example 8

### Prophetic

### Producing Isobutanol Using Strain with acrA or acrB Mutation

E. coli strains engineered to express an isobutanol biosynthetic pathway are described in commonly owned and co-pending US patent application publication US20070092957A1, Examples 9-15, which are herein incorporated by reference. Strain BL21 (DE) 1.5GI yqhD/pTrc99a::budB-11vC-ilvD-kivD was derived from BL21 (DE3) (Invitrogen) and was engineered to contain an operon expressed from the trc promoter that includes the Klebsiella pneumoniae budB coding region for acetolactate synthase, the E. coli ilvC coding region for acetohydroxy acid reductoisomerase, the E. coli ilvD coding region for acetohydroxy acid dehydratase and the Lactococcus lactis kivD coding region for branched chain α-keto acid decarboxylase. In addition, in this strain the native promoter of the yqhD gene (encoding 1,3-propanediol dehydrogenase) was replaced with the 1.5GI promoter (WO 2003/089621). The same promoter replacement was made in E. coli strain MG1655 to create MG1655 1.5GI-yqhD::Cm, and the same plasmid was introduced resulting in strain MG655 1.5/GI yqhD/pTrc99A::budB-11vC-ilvD-kivD. These isobutanol pathway containing strains are engineered for butanol tolerance by introducing a modification in either the acrA or the acrB genes. The strains are transduced to Kanamycin resistance with 2 distinct phage P1 lysates (either P1ᵥᵢᵣ or P1clr100Cam can be used). To make one lysate, for inactivating the acrB gene, phage are grown on one of the acrB strains isolated by transposon mutagenesis of strain EC100 described above (DPD1852 or DPD1858) or the Keio collection mutant JW0451. For the second lysate, phage are grown on strain JW0452 (acrA) of the Keio collection to package DNA for introducing the other mutation to be introduced, acrA::kan. Kanamycin resistance is selected on agar solidified LB medium using 50 μg/ml of the antibiotic. The resultant transductants have null mutations in the genes (acrB::kan, acrB::Tn, acrA::kan).

Separately, an isobutanol biosynthetic pathway and butanol tolerance are engineered in the same strain by adding the isobutanol pathway to acrB or acrA mutated strains. EC100 acrB::Tn (DPD1852 or DPD1858) and BW25113 acrA::kan (JW0452), acrB::kan (JW0451), along with EC100 and BW25113 controls, are transduced to chloramphenicol resistance with a phage P1 lysate of E. coli MG1655 1.5GI yqhD::Cm to replace the yqhD promoter with the 1.5GI promoter. The resulting strains are transformed with pTrc99A::budB-11vC-ilvD-kivD yielding pTrc99A::budB-11vC-ilvD-kivD/EC100 1.5GI yqhD::Cm, pTrc99A::budB-11vC-ilvD-kivD/EC100 spoT::Tn 1.5GI yqhD::Cm, pTrc99A::budB-11vC-ilvD-kivD/BW25113 1.5GI yqhD::Cm and pTrc99A::budB-11vC-ilvD-kivD/BW25113 rpoZ::kan 1.5GI yqhD::Cm. These strains in the MG1655, EC100 and BW25113 backgrounds are analyzed for butanol production.

The cells from cultures or each strain are used to inoculate shake flasks (approximately 175 mL total volume) containing 50 or 170 mL of TM3a/glucose medium (with appropriate antibiotics) to represent high and low oxygen conditions, respectively. TM3a/glucose medium contains (per liter): glucose (10 g), KH₂PO₄ (13.6 g), citric acid monohydrate (2.0 g), (NH₄)₂SO₄ (3.0 g), MgSO₄.7H₂O (2.0 g), CaCl₂.2H₂O (0.2 g), ferric ammonium citrate (0.33 g), thiamine HCl (1.0 mg), yeast extract (0.50 g), and 10 mL of trace elements solution. The pH was adjusted to 6.8 with NH₄OH. The trace elements solution contains: citric acid .H₂O (4.0 g/L), MnSO₄.H₂O (3.0 g/L), NaCl (1.0 g/L), FeSO₄.7H₂O (0.10 g/L), COCl₂.6H₂O (0.10 g/L), ZnSO₄. 7H₂O (0.10 g/L), CuSO₄.5H₂O (0.010 g/L), H₃BO₃ (0.010 g/L), and Na₂MoO₄.2H₂O (0.010 g/L).

The flasks are inoculated at a starting OD₆₀₀ of ≦0.01 units and incubated at 34° C. with shaking at 300 rpm. The flasks containing 50 mL of medium are closed with 0.2 μm filter caps; the flasks containing 150 mL of medium are closed with sealed caps. IPTG is added to a final concentration of 0.04 mM when the cells reach an OD₆₀₀ of ≧0.4 units. Approximately 18 h after induction, an aliquot of the broth is analyzed by HPLC (Shodex Sugar SH1011 column (Showa Denko America, Inc. NY) with refractive index (RI) detection) and GC (Varian CP-WAX 58(FFAP) CB, 0.25 mm×0.2 μm×25 m (Varian, Inc., Palo Alto, Calif.) with flame ionization detection (FID)) for isobutanol content, as described in the General Methods section. No isobutanol is detected in control strains. Molar selectivities and titers of isobutanol produced by strains carrying pTrc99A::budB-11vC-ilvD-kivD are obtained. Significantly higher titers of isobutanol are obtained in the spoT and rpoZ cultures than in the parental strains.

### Example 9

### Prophetic

### Producing 2-butanol Using Strain with acrA or acrB Mutation

The engineering of E. coli for expression of a 2-butanol biosynthetic pathway is described in commonly owned and co-pending US Patent Application Publication US20070259410A1, Examples 6 and 7, which are herein incorporated by reference. Construction is described of two plasmids for upper and lower pathway expression. In pBen-budABC, an NPR promoter (Bacillus amyloliquefaciens neutral protease promoter) directs expression of Klebsiella pneumoniae budABC coding regions for acetolactate decarboxylase, acetolactate synthase, and butanediol dehydrogenase. In pBen-pdd-sadh an NPR promoter directs expression of Klebsiella oxytoca pddABC coding regions for butanediol dehydratase alpha subunit, butanediol dehydratase beta subunit, and butanediol dehydratase gamma subunit, and the Rhodococcus ruber sadh coding region for butanol dehydrogenase. Plasmid p2BOH is described containing both operons, and strain NM522/p2BOH containing this plasmid for 2-butanol pathway expression is described.

The NM522/p2BOH strain is engineered for butanol tolerance by introducing a modification in either the acrA gene or the acrB gene. The strain is transduced to kanamycin resistance with 2 distinct P1 lysates (either P1 vir or P1clr100Cam can be used). To make one lysate, for inactivating the acrB gene, phage are grown on one of the acrB::Tn strains isolated by transposon mutagenesis of strain EC100 described above in Example 2 (DPD1852 or DPD1858). For the second lysate, phage are grown on strain JW0452 of the Keio collection to pick up DNA for introducing the other mutation, acrA::kan. Kanamycin resistance is selected on agar solidified LB medium using 50 μg/ml of the antibiotic. The resultant transductants have null mutations, acrB::Tn and acrA::kan, and are called NM522 acrB::Tn/p2BOH and NM522 acrA::kan/p2BOH.

E. coli NM522/p2BOH, NM522 acrB::Tn/p2BOH and NM522 acrA::kan/p2BOH are inoculated into a 250 mL shake flask containing 50 mL of medium and shaken at 250 rpm and 35° C. The medium is composed of: dextrose, 5 g/L; MOPS, 0.05 M; ammonium sulfate, 0.01 M; potassium phosphate, monobasic, 0.005 M; S10 metal mix, 1% (v/v); yeast extract, 0.1% (w/v); casamino acids, 0.1% (w/v); thiamine, 0.1 mg/L; proline, 0.05 mg/L; and biotin 0.002 mg/L, and is titrated to pH 7.0 with KOH. S10 metal mix contains: MgCl₂, 200 mM; CaCl₂, 70 mM; MnCl₂, 5 mM; FeCl₃, 0.1 mM; ZnCl₂, 0.1 mM; thiamine hydrochloride, 0.2 mM; CuSO₄, 172 μM; COCl₂, 253 μM; and Na₂MoO₄, 242 μM. After 18 h, 2-butanol is detected by HPLC or GC analysis using methods that are well known in the art, for example, as described in the General Methods section above. Higher titers are obtained from the acrA and acrB derivatives.

### Example 10

### Prophetic

### Producing 1-butanol Using Strain with acrA and/or acrB Mutations

E. coli strains engineered to express a 1-butanol biosynthetic pathway are described in commonly owned and co-pending US Patent Application Publication US20080182308A1, Example 13, which is herein incorporated by reference. Two plasmids were constructed that carry genes encoding the 1-butanol pathway. Plasmid PBHR T7-ald contains a gene for expression of butyraldehyde dehydrogenase (ald). Plasmid pTrc99a-E-C-H-T contains a four gene operon comprising the upper pathway, for expression of acetyl-CoA acetyltransferase (thlA), 3-hydroxybutyryl-CoA dehydrogenase (hbd), crotonase (crt), and butyryl-CoA dehydrogenase (trans-2-enoyl-CoA reductase, EgTER(opt)) (EgTER(opt), crt, hbd and thlA). In addition, in this strain the native promoter of the yqhD gene (encoding 1,3-propanediol dehydrogenase) was replaced with the 1.5GI promoter (WO 2003/089621).

All genes of this 1-butanol pathway are combined with null acrA and acrB mutations for increased butanol tolerance as follows. EC100 acrB::Tn (DPD1852 or DPD1858) and BW25113 acrA::kan (JW0452), along with EC100 and BW25113 controls, are transduced to chloramphenicol resistance with a phage P1 lysate of E. coli MG1655 1.5GI yqhD::Cm to replace the yqhD promoter with the 1.5GI promoter. The resulting strains are transformed with PBHR T7-ald and pTrc99a-E-C-H-T producing engineered strains with the 1-butaonl biosynthetic pathway.

Strains containing the 1-butanol pathway and butanol tolerance are also constructed by introducing a modified acrA gene or acrB gene into 1-butanol pathway containing strains. Construction of E. coli strain MG1655 (DE3) 1.5GI-yqhD::Cm/pTrc99a-E-C-H-T/PBHR T7-ald was also described in US Patent Application Publication US20080182308A1 Example 13. This strain was then modified to introduce acrA and acrB alleles by generalized transduction with phage P1. The transformants were transduced to kanamycin resistance with 2 distinct phage P1 lysates (either P1 vir or P1clr100Cam can be used). To make one lysate, for inactivating the acrB gene, phage are grown on one of the acrB::Tn strains isolated by transposon mutagenesis of strain EC100 described above in Example 2 (DPD1852 or DPD1858). For the second lysate, phage are grown on strain JW0452 of the Keio collection to pickup DNA for introducing the acrA::kan mutation. Kanamycin resistance is selected on agar solidified LB medium using 50 μg/ml of the antibiotic. The resultant transductants have no AcrA or AcrB activity in the MG1655 background.

The transductants from the MG1655 background and the transformants from the EC100 and BW25113 backgrounds are used to inoculate shake flasks (approximately 175 mL total volume) containing 15, 50 and 150 mL of TM3a/glucose medium (with appropriate antibiotics) to represent high, medium and low oxygen conditions, respectively. TM3a/glucose medium contains (per liter): 10 g glucose, 13.6 g KH₂PO₄, 2.0 g citric acid monohydrate, 3.0 g (NH₄)₂SO₄, 2.0 g MgSO₄.7H₂O, 0.2 g CaCl₂. 2H₂O, 0.33 g ferric ammonium citrate, 1.0 mg thiamine HCl, 0.50 g yeast extract, and 10 mL trace elements solution, adjusted to pH 6.8 with NH₄OH. The solution of trace elements contains: citric acid .H₂O (4.0 g/L), MnSO₄. H₂O (3.0 g/L), NaCl (1.0 g/L), FeSO₄.7H₂O (0.10 g/L), COCl₂.6H₂O (0.10 g/L), ZnSO₄.7H₂O (0.10 g/L), CuSO₄.5H₂O (0.010 g/L), H₃BO₃ (0.010 g/L), and Na₂MoO₄.2H₂O (0.010 g/L). The flasks are inoculated at a starting OD₆₀₀ of ≦0.01 units and incubated at 34° C. with shaking at 300 rpm. The flasks containing 15 and 50 mL of medium are capped with vented caps; the flasks containing 150 mL, are capped with non-vented caps to minimize air exchange. IPTG is added to a final concentration of 0.04 mM; the OD₆₀₀ of the flasks at the time of addition is ≧0.4 units. Approximately 15 h after induction, an aliquot of the broth is analyzed by HPLC (Shodex Sugar SH1011 column) with refractive index (RI) detection and GC (Varian CP-WAX 58(FFAP) CB column, 25 m×0.25 mm id×0.2 μm film thickness) with flame ionization detection (FID) for 1-butanol content, as described in the General Methods section. Titers of 1-butanol are found to be higher in strains harboring either the acrA or acrB alleles.

What is claimed is:

1. A recombinant Escherichia coli cell producing butanol or 2-butanone said E. coli cell comprising at least one genetic modification which reduces production of a protein selected from the group consisting of AcrA and AcrB.

2. The E. coli cell of claim 1 comprising a recombinant biosynthetic pathway selected from the group consisting of:
a) a 1-butanol biosynthetic pathway;
b) a 2-butanol biosynthetic pathway;
c) an isobutanol biosynthetic pathway; and
d) a 2-butanone biosynthetic pathway.

3. The E. coli cell of claim 1, wherein the at least one genetic modification is a disruption in a endogenous gene selected from the group consisting of acrA and acrB gene.

4. The E. coli cell of claim 1, additionally comprising at least one genetic modification which reduces accumulation of (p)ppGpp.

5. The E. coli cell of claim 4, wherein the at least one genetic modification which reduces accumulation of (p)ppGpp reduces production of SpoT or RelA.

6. The E. coli cell of claim 5, wherein the at least one genetic modification which reduces accumulation of (p)ppGpp is a disruption in an endogenous gene selected from the group consisting of spoT and re/A or in an operon comprising an open reading frame encoding SpoT or RelA.

7. The E. coli cell of claim 4, wherein the genetic modification reduces (p)ppGpp synthetic activity of encoded endogenous SpoT protein.

8. The E. coli cell of claim 4, wherein the genetic modification increases (p)ppGpp degradative activity by increasing expression of a SpoT with reduced (p)ppGpp synthetic activity.

9. The recombinant E. coli cell of claim 2 wherein the 1-butanol biosynthetic pathway comprises:
a) at least one genetic construct encoding an acetyl-CoA acetyltransferase;
b) at least one genetic construct encoding 3-hydroxybutyryl-CoA dehydrogenase;
c) at least one genetic construct encoding crotonase;
d) at least one genetic construct encoding butyryl-CoA dehydrogenase;
e) at least one genetic construct encoding butyraldehyde; dehydrogenase; and
f) at least one genetic construct encoding 1-butanol dehydrogenase.

10. The recombinant E. coli cell of claim 2 wherein the 2-butanol biosynthetic pathway comprises:
a) at least one genetic construct encoding an acetolactate synthase;
b) at least one genetic construct encoding acetolactate decarboxylase;
c) at least one genetic construct encoding butanediol dehydrogenase;
d) at least one genetic construct encoding butanediol dehydratase; and
e) at least one genetic construct encoding 2-butanol dehydrogenase.

11. The recombinant E. coli cell of claim 2 wherein the isobutanol biosynthetic pathway comprises:
a) at least one genetic construct encoding an acetolactate synthase;
b) at least one genetic construct encoding acetohydroxy acid isomeroreductase;
c) at least one genetic construct encoding acetohydroxy acid dehydratase;
d) at least one genetic construct encoding branched-chain keto acid decarboxylase; and
e) at least one genetic construct encoding branched-chain alcohol dehydrogenase.

12. The recombinant E. coli cell of claim 2 wherein the 2-butanone biosynthetic pathway comprises:
a) at least one genetic construct encoding an acetolactate synthase;
b) at least one genetic construct encoding acetolactate decarboxylase;
c) at least one genetic construct encoding butanediol dehydrogenase; and
d) at least one genetic construct encoding butanediol dehydratase.

13. A process for generating the E. coli host cell of claim 1 comprising:
a) providing a recombinant bacterial host cell producing butanol or 2-butanone; and
b) creating at least one genetic modification which redues production of AcrA or AcrB, or both AcrA and AcrB proteins.

14. A process for production of butanol or 2-butanone from a recombinant E. coli cell comprising:
(a) providing a recombinant E. coli cell which
1) produces butanol or 2-butanone and
2) comprises at least one genetic modification which reduces production of AcrA or AcrB, or both AcrA and AcrB; and
(b) culturing the strain of (a) under conditions wherein butanol or 2-butanone is produced.

15. The process according to claim 14, wherein the recombinant E. coli comprises a biosynthetic pathway selected from the group consisting of:
a) a 1-butanol biosynthetic pathway;
b) a 2-butanol biosynthetic pathway;
c) an isobutanol biosynthetic pathway; and
d) a 2-butanone biosynthetic pathway

16. The process according to claim 14, wherein the recombinant E. coli cell additionally comprises at least one genetic modification which reduces accumulation of (p)ppGpp.

17. The process according to claim 16, wherein the at least one genetic modification which reduces accumulation of (p)ppGpp reduces production of SpoT or RelA.

18. The process according to claim 17, wherein the at least one genetic modification which reduces accumulation of (p)ppGpp is a disruption in an endogenous gene selected from the group consisting of spoT and re/A or in an operon comprising an open reading frame encoding SpoT or RelA.

19. The process according to claim 17, wherein the genetic modification reduces (p)ppGpp synthetic activity of encoded endogenous SpoT protein.

20. The process according to claim 17, wherein the genetic modification increases (p)ppGpp degradative activity by increasing expression of a SpoT with reduced (p)ppGpp synthetic activity.

21. The process according to claim 15, wherein the 1-butanol biosynthetic pathway comprises:
a) at least one genetic construct encoding an acetyl-CoA acetyltransferase;
b) at least one genetic construct encoding 3-hydroxybutyryl-CoA dehydrogenase;
c) at least one genetic construct encoding crotonase;
d) at least one genetic construct encoding butyryl-CoA dehydrogenase;
e) at least one genetic construct encoding butyraldehyde;
dehydrogenase; and
f) at least one genetic construct encoding 1-butanol dehydrogenase.

22. The process according to claim 15, wherein the 2-butanol biosynthetic pathway comprises:
a) at least one genetic construct encoding an acetolactate synthase;
b) at least one genetic construct encoding acetolactate decarboxylase;
c) at least one genetic construct encoding butanediol dehydrogenase;
d) at least one genetic construct encoding butanediol dehydratase; and
e) at least one genetic construct encoding 2-butanol dehydrogenase.

23. The process according to claim 15, wherein the isobutanol biosynthetic pathway comprises:
a) at least one genetic construct encoding an acetolactate synthase;
b) at least one genetic construct encoding acetohydroxy acid isomeroreductase;
c) at least one genetic construct encoding acetohydroxy acid dehydratase;
d) at least one genetic construct encoding branched-chain keto acid decarboxylase; and
e) at least one genetic construct encoding branched-chain alcohol dehydrogenase.

24. The process according to claim 15, wherein the 2-butanone biosynthetic pathway comprises:
a) at least one genetic construct encoding an acetolactate synthase;
b) at least one genetic construct encoding acetolactate decarboxylase;
c) at least one genetic construct encoding butanediol dehydrogenase; and
d) at least one genetic construct encoding butanediol dehydratase.


SEQUENCE LISTING (73 sequences)

SEQ ID NO: 1 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 2 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 3 - DNA, 1179 bp, Clostridium acetobutylicum
atgagagatgtagtaatagtaagtgctgtaagaactgcaataggagcatatggaaaaacattaaaggatgtacctgcaacagagttaggagctatagtaataaaggaagctgtaagaagagctaatataaatccaaatgagattaatgaagttatttttggaaatgtacttcaagctggattaggccaaaacccagcaagacaagcagcagtaaaagcaggattacctttagaaacacctgcgtttacaatcaataaggtttgtggttcaggtttaagatctataagtttagcagctcaaattataaaagctggagatgctgataccattgtagtaggtggtatggaaaatatgtctagatcaccatatttgattaacaatcagagatggggtcaaagaatgggagatagtgaattagttgatgaaatgataaaggatggtttgtgggatgcatttaatggatatcatatgggagtaactgcagaaaatattgcagaacaatggaatataacaagagaagagcaagatgaattttcacttatgtcacaacaaaaagctgaaaaagccattaaaaatggagaatttaaggatgaaatagttcctgtattaataaagactaaaaaaggtgaaatagtctttgatcaagatgaatttcctagattcggaaacactattgaagcattaagaaaacttaaacctattttcaaggaaaatggtactgttacagcaggtaatgcatccggattaaatgatggagctgcagcactagtaataatgagcgctgataaagctaacgctctcggaataaaaccacttgctaagattacttcttacggatcatatggggtagatccatcaataatgggatatggagctttttatgcaactaaagctgccttagataaaattaatttaaaacctgaagacttagatttaattgaagctaacgaggcatatgcttctcaaagtatagcagtaactagagatttaaatttagatatgagtaaagttaatgttaatggtggagctatagcacttggacatccaataggtgcatctggtgcacgtattttagtaacattactatacgctatgcaaaaaagagattcaaaaaaaggtcttgctactctatgtattggtggaggtcagggaacagctctcgtagttgaaagagactaa

SEQ ID NO: 4 - protein, 392 aa, Clostridium acetobutylicum
MRDVVIVSAVRTAIGAYGKTLKDVPATELGAIVIKEAVRRANINPNEINEVIFGNVLQAGLGQNPARQAAVKAGLPLETPAFTINKVCGSGLRSISLAAQIIKAGDADTIVVGGMENMSRSPYLINNQRWGQRMGDSELVDEMIKDGLWDAFNGYHMGVTAENIAEQWNITREEQDEFSLMSQQKAEKAIKNGEFKDEIVPVLIKTKKGEIVFDQDEFPRFGNTIEALRKLKPIFKENGTVTAGNASGLNDGAAALVIMSADKANALGIKPLAKITSYGSYGVDPSIMGYGAFYATKAALDKINLKPEDLDLIEANEAYASQSIAVTRDLNLDMSKVNVNGGAIALGHPIGASGARILVTLLYAMQKRDSKKGLATLCIGGGQGTALVVERD

SEQ ID NO: 5 - DNA, 849 bp, Clostridium acetobutylicum
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 6 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 7 - DNA, 786 bp, Clostridium acetobutylicum
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 8 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 9 - DNA, 1197 bp, Clostridium acetobutylicum
atgatagtaaaagcaaagtttgtaaaaggatttatcagagatgtacatccttatggttgcagaagggaagtactaaatcaaatagattattgtaagaaggctattgggtttaggggaccaaagaaggttttaattgttggagcctcatctgggtttggtcttgctactagaatttcagttgcatttggaggtccagaagctcacacaattggagtatcctatgaaacaggagctacagatagaagaataggaacagcgggatggtataataacatattttttaaagaatttgctaaaaaaaaaggattagttgcaaaaaacttcattgaggatgccttttctaatgaaaccaaagataaagttattaagtatataaaggatgaatttggtaaaatagatttatttgtttatagtttagctgcgcctaggagaaaggactataaaactggaaatgtttatacttcaagaataaaaacaattttaggagattttgagggaccgactattgatgttgaaagagacgagattactttaaaaaaggttagtagtgctagcattgaagaaattgaagaaactagaaaggtaatgggtggagaggattggcaagagtggtgtgaagagctgctttatgaagattgtttttcggataaagcaactaccatagcatactcgtatataggatccccaagaacctacaagatatatagagaaggtactataggaatagctaaaaaggatcttgaagataaggctaagcttataaatgaaaaacttaacagagttataggtggtagagcctttgtgtctgtgaataaagcattagttacaaaagcaagtgcatatattccaacttttcctctttatgcagctattttatataaggtcatgaaagaaaaaaatattcatgaaaattgtattatgcaaattgagagaatgttttctgaaaaaatatattcaaatgaaaaaatacaatttgatgacaagggaagattaaggatggacgatttagagcttagaaaagacgttcaagacgaagttgatagaatatggagtaatattactcctgaaaattttaaggaattatctgattataagggatacaaaaaagaattcatgaacttaaacggttttgatctagatggggttgattatagtaaagacctggatatagaattattaagaaaattagaaccttaa

SEQ ID NO: 10 - protein, 398 aa, Clostridium acetobutylicum
MIVKAKFVKGFIRDVHPYGCRREVLNQIDYCKKAIGFRGPKKVLIVGASSGFGLATRISVAFGGPEAHTIGVSYETGATDRRIGTAGWYNNIFFKEFAKKKGLVAKNFIEDAFSNETKDKVIKYIKDEFGKIDLFVYSLAAPRRKDYKTGNVYTSRIKTILGDFEGPTIDVERDEITLKKVSSASIEEIEETRKVMGGEDWQEWCEELLYEDCFSDKATTIAYSYIGSPRTYKIYREGTIGIAKKDLEDKAKLINEKLNRVIGGRAFVSVNKALVTKASAYIPTFPLYAAILYKVMKEKNIHENCIMQIERMFSEKIYSNEKIQFDDKGRLRMDDLELRKDVQDEVDRIWSNITPENFKELSDYKGYKKEFMNLNGFDLDGVDYSKDLDIELLRKLEP

SEQ ID NO: 11 - DNA, 1407 bp, Clostridium beijerinckii
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 12 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 13 - DNA, 1215 bp, Clostridium acetobutylicum
atggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaaaacgcctccgaagtcctacaaatattcaaaaaatctgtgtaa

SEQ ID NO: 14 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 15 - DNA, 1170 bp, Clostridium acetobutylicum
atgctaagttttgattattcaataccaactaaagttttttttggaaaaggaaaaatagacgtaattggagaagaaattaagaaatatggctcaagagtgcttatagtttatggcggaggaagtataaaaaggaacggtatatatgatagagcaacagctatattaaaagaaaacaatatagctttctatgaactttcaggagtagagccaaatcctaggataacaacagtaaaaaaaggcatagaaatatgtagagaaaataatgtggatttagtattagcaatagggggaggaagtgcaatagactgttctaaggtaattgcagctggagtttattatgatggcgatacatgggacatggttaaagatccatctaaaataactaaagttcttccaattgcaagtatacttactctttcagcaacagggtctgaaatggatcaaattgcagtaatttcaaatatggagactaatgaaaagcttggagtaggacatgatgatatgagacctaaattttcagtgttagatcctacatatacttttacagtacctaaaaatcaaacagcagcgggaacagctgacattatgagtcacacctttgaatcttactttagtggtgttgaaggtgcttatgtgcaggacggtatagcagaagcaatcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttattaa

SEQ ID NO: 16 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 17 - DNA, 780 bp, Klebsiella pneumoniae
atgaatcattctgctgaatgcacctgcgaagagagtctatgcgaaaccctgcgggcgttttccgcgcagcatcccgagagcgtgctctatcagacatcgctcatgagcgccctgctgagcggggtttacgaaggcagcaccaccatcgcggacctgctgaaacacggcgatttcggcctcggcacctttaatgagctggacggggagctgatcgccttcagcagtcaggtctatcagctgcgcgccgacggcagcgcgcgcaaagcccagccggagcagaaaacgccgttcgcggtgatgacctggttccagccgcagtaccggaaaacctttgaccatccggtgagccgccagcagctgcacgaggtgatcgaccagcaaatcccctctgacaacctgttctgcgccctgcgcatcgacggccatttccgccatgcccatacccgcaccgtgccgcgccagacgccgccgtaccgggcgatgaccgacgtcctcgacgatcagccggtgttccgctttaaccagcgcgaaggggtgctggtcggcttccggaccccgcagcatatgcaggggatcaacgtcgccgggtatcacgagcactttattaccgatgaccgcaaaggcggcggtcacctgctggattaccagctcgaccatggggtgctgaccttcggcgaaattcacaagctgatgatcgacctgcccgccgacagcgcgttcctgcaggctaatctgcatcccgataatctcgatgccgccatccgttccgtagaaagttaa

SEQ ID NO: 18 - protein, 259 aa, Klebsiella pneumoniae
MNHSAECTCEESLCETLRAFSAQHPESVLYQTSLMSALLSGVYEGSTTIADLLKHGDFGLGTFNELDGELIAFSSQVYQLRADGSARKAQPEQKTPFAVMTWFQPQYRKTFDHPVSRQQLHEVIDQQIPSDNLFCALRIDGHFRHAHTRTVPRQTPPYRAMTDVLDDQPVFRFNQREGVLVGFRTPQHMQGINVAGYHEHFITDDRKGGGHLLDYQLDHGVLTFGEIHKLMIDLPADSAFLQANLHPDNLDAAIRSVES

SEQ ID NO: 19 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaattgacaaggtcttcgactcactgctggattcctcgattcgcattattccggtacgccacgaagccaacgccgcgtttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccgtcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctgaaccagtttgccctgcatccgctgcgcatcgttcgcgccatgcaggacatcgtcaacagcgacgtcacgttgaccgtggacatgggcagcttccatatctggattgcccgctacctgtacagcttccgcgcccgtcaggtgatgatctccaacggccagcagaccatgggcgtcgccctgccctgggctatcggcgcctggctggtcaatcctgagcgaaaagtggtctccgtctccggcgacggcggcttcctgcagtcgagcatggagctggagaccgccgtccgcctgaaagccaacgtactgcacctgatctgggtcgataacggctacaacatggtggccattcaggaagagaaaaaataccagcgcctgtccggcgtcgagttcgggccgatggattttaaagcctatgccgaatccttcggcgcgaaagggtttgccgtggaaagcgccgaggcgctggagccgaccctgcacgcggcgatggacgtcgacggcccggcggtggtggccattccggtggattatcgcgataacccgctgctgatgggccagctgcatctgagtcagattctgtaa

SEQ ID NO: 20 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAVEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYSFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLHAAMDVDGPAVVAIPVDYRDNPLLMGQLHLSQIL

SEQ ID NO: 21 - DNA, 771 bp, Klebsiella pneumoniae
atgaaaaaagtcgcacttgttaccggcgccggccaggggattggtaaagctatcgcccttcgtctggtgaaggatggatttgccgtggccattgccgattataacgacgccaccgccaaagcggtcgcctcggaaatcaaccaggccggcggacacgccgtggcggtgaaagtggatgtctccgaccgcgatcaggtatttgccgccgttgaacaggcgcgcaaaacgctgggcggcttcgacgtcatcgtcaataacgccggtgtggcaccgtctacgccgatcgagtccattaccccggagattgtcgacaaagtctacaacatcaacgtcaaaggggtgatctggggtattcaggcggcggtcgaggcctttaagaaagaggggcacggcgggaaaatcatcaacgcctgttcccaggccggccacgtcggcaacccggagctggcggtgtatagctccagtaaattcgcggtacgcggcttaacccagaccgccgctcgcgacctcgcgccgctgggcatcacggtcaacggctactgcccggggattgtcaaaacgccaatgtgggccgaaattgaccgccaggtgtccgaagccgccggtaaaccgctgggctacggtaccgccgagttcgccaaacgcatcactctcggtcgtctgtccgagccggaagatgtcgccgcctgcgtctcctatcttgccagcccggattctgattacatgaccggtcagtcgttgctgatcgacggcgggatggtatttaactaa

SEQ ID NO: 22 - protein, 256 aa, Klebsiella pneumoniae
MKKVALVTGAGQGIGKAIALRLVKDGFAVAIADYNDATAKAVASEINQAGGHAVAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 23 - DNA, 1665 bp, Klebsiella oxytoca
atgagatcgaaaagatttgaagcactggcgaaacgccctgtgaatcaggacggcttcgttaaggagtggatcgaagaaggctttatcgcgatggaaagcccgaacgacccaaaaccgtcgattaaaatcgttaacggcgcggtgaccgagctggacgggaaaccggtaagcgattttgacctgatcgaccactttatcgcccgctacggtatcaacctgaaccgcgccgaagaagtgatggcgatggattcggtcaagctggccaacatgctgtgcgatccgaacgttaaacgcagcgaaatcgtcccgctgaccaccgcgatgacgccggcgaaaattgtcgaagtggtttcgcatatgaacgtcgtcgagatgatgatggcgatgcagaaaatgcgcgcccgccgcaccccgtcccagcaggcgcacgtcaccaacgtcaaagataacccggtacagattgccgccgacgccgccgaaggggcatggcgcggatttgacgaacaggaaaccaccgttgcggtagcgcgctatgcgccgttcaacgccatcgcgctgctggtgggctcgcaggtaggccgtccgggcgtgctgacgcagtgctcgctggaagaagccaccgagctgaagctcggcatgctgggccacacctgctacgccgaaaccatctccgtctacggcaccgagccggtctttaccgacggcgacgacacgccgtggtcgaagggcttcctcgcctcgtcctacgcctctcgcgggctgaaaatgcgctttacctccggctccggctcggaagtgcagatgggctacgccgaaggcaaatccatgctttatctggaagcgcgctgcatctacatcaccaaagccgcgggcgtacagggtctgcaaaacggttccgtaagctgcatcggcgtgccgtctgcggtgccttccggcattcgcgcggtgctggcggaaaacctgatctgttcgtcgctggatctggagtgcgcctccagcaacgaccagaccttcacccactccgatatgcgtcgtaccgcgcgcctgctgatgcagttcctgccgggcaccgactttatctcctccggttattccgcggtgccgaactacgacaacatgttcgccggctccaacgaagatgccgaagactttgacgactacaacgtcatccagcgcgacctgaaggtggacggcggtttgcgtccggttcgcgaagaggacgtcatcgccatccgtaacaaagccgcccgcgcgctgcaggccgtgtttgccggaatggggctgccgccgattaccgatgaagaagttgaagccgcgacctacgcccacggttcgaaagatatgccggagcgcaacatcgtcgaagacatcaagttcgcccaggaaatcatcaataaaaaccgcaacggtctggaagtggtgaaagcgctggcgcagggcggattcaccgacgtggcccaggacatgctcaacatccagaaagctaagctgaccggggactacctgcatacctccgcgattatcgtcggcgacgggcaggtgctgtcagccgtcaacgacgtcaacgactatgccggtccggcaacgggctatcgcctgcagggcgaacgctgggaagagattaaaaacatccctggcgctcttgatcccaacgagattgattaa

SEQ ID NO: 24 - protein, 554 aa, Klebsiella oxytoca
MRSKRFEALAKRPVNQDGFVKEWIEEGFIAMESPNDPKPSIKIVNGAVTELDGKPVSDFDLIDHFIARYGINLNRAEEVMAMDSVKLANMLCDPNVKRSEIVPLTTAMTPAKIVEVVSHMNVVEMMMAMQKMRARRTPSQQAHVTNVKDNPVQIAADAAEGAWRGFDEQETTVAVARYAPFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSCIGVPSAVPSGIRAVLAENLICSSLDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAQGGFTDVAQDMLNIQKAKLTGDYLHTSAIIVGDGQVLSAVNDVNDYAGPATGYRLQGERWEEIKNIPGALDPNEID

SEQ ID NO: 25 - DNA, 675 bp, Klebsiella oxytoca
atggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttga

SEQ ID NO: 26 - protein, 224 aa, Klebsiella oxytoca
MEINEKLLRQIIEDVLSEMKGSDKPVSFNAPAASAAPQATPPAGDGFLTEVGEARQGTQQDEVIIAVGPAFGLAQTVNIVGIPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 27 - DNA, 522 bp, Klebsiella oxytoca
atgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaa

SEQ ID NO: 28 - protein, 173 aa, Klebsiella oxytoca
MNTDAIESMVRDVLSRMNSLQGEAPAAAPAAGGASRSARVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLESRYQAKICAAFVREAATLYVERKKLKGDD

SEQ ID NO: 29 - DNA, 1041 bp, Rhodococcus ruber
atgaaagccctccagtacaccgagatcggctccgagccggtcgtcgtcgacgtccccaccccggcgcccgggccgggtgagatcctgctgaaggtcaccgcggccggcttgtgccactcggacatcttcgtgatggacatgccggcagagcagtacatctacggtcttcccctcaccctcggccacgagggcgtcggcaccgtcgccgaactcggcgccggcgtcaccggattcgagacgggggacgccgtcgccgtgtacgggccgtgggggtgcggtgcgtgccacgcgtgcgcgcgcggccgggagaactactgcacccgcgccgccgagctgggcatcaccccgcccggtctcggctcgcccgggtcgatggccgagtacatgatcgtcgactcggcgcgccacctcgtcccgatcggggacctcgaccccgtcgcggcggttccgctcaccgacgcgggcctgacgccgtaccacgcgatctcgcgggtcctgcccctgctgggacccggctcgaccgcggtcgtcatcggggtcggcggactcgggcacgtcggcatccagatcctgcgcgccgtcagcgcggcccgcgtgatcgccgtcgatctcgacgacgaccgactcgcgctcgcccgcgaggtcggcgccgacgcggcggtgaagtcgggcgccggggcggcggacgcgatccgggagctgaccggcggtgagggcgcgacggcggtgttcgacttcgtcggcgcccagtcgacgatcgacacggcgcagcaggtggtcgcgatcgacgggcacatctcggtggtcggcatccatgccggcgcccacgccaaggtcggcttcttcatgatcccgttcggcgcgtccgtcgtgacgccgtactggggcacgcggtccgagctgatggacgtcgtggacctggcccgtgccggccggctcgacatccacaccgagacgttcaccctcgacgagggacccacggcctaccggcggctacgcgagggcagcatccgcggccgcggggtggtcgtcccgggctga

SEQ ID NO: 30 - protein, 346 aa, Rhodococcus ruber
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDLDDDRLALAREVGADAAVKSGAGAADAIRELTGGEGATAVFDFVGAQSTIDTAQQVVAIDGHISVVGIHAGAHAKVGFFMIPFGASVVTPYWGTRSELMDVVDLARAGRLDIHTETFTLDEGPTAYRRLREGSIRGRGVVVPG

SEQ ID NO: 31 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 32 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 33 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 34 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 35 - DNA, 1662 bp, Lactococcus lactis
tctagacatatgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaaggatcc

SEQ ID NO: 36 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 37 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 38 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 39 - DNA, 1194 bp, Escherichia coli K12
atgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcgacatacgacagtgcgaaaggtgatctggcgaaagcccaggctgcagccaatatcgcgcaattgacggtgaatcgttatcagaaactgctcggtactcagtacatcagtaagcaagagtacgatcaggctctggctgatgcgcaacaggcgaatgctgcggtaactgcggcgaaagctgccgttgaaactgcgcggatcaatctggcttacaccaaagtcacctctccgattagcggtcgcattggtaagtcgaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcactgatcaccagtgacggcattaagttcccgcaggacggtacgctggaattctctgacgttaccgttgatcagaccactgggtctatcaccctacgcgctatcttcccgaacccggatcacactctgctgccgggtatgttcgtgcgcgcacgtctggaagaagggcttaatccaaacgctattttagtcccgcaacagggcgtaacccgtacgccgcgtggcgatgccaccgtactggtagttggcgcggatgacaaagtggaaacccgtccgatcgttgcaagccaggctattggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtcttaa

SEQ ID NO: 40 - protein, 397 aa, Escherichia coli K12
MNKNRGFTPLAVVLMLSGSLALTGCDDKQAQQGGQQMPAVGVVTVKTEPLQITTELPGRTSAYRIAEVRPQVSGIILKRNFKEGSDIEAGVSLYQIDPATYQATYDSAKGDLAKAQAAANIAQLTVNRYQKLLGTQYISKQEYDQALADAQQANAAVTAAKAAVETARINLAYTKVTSPISGRIGKSNVTEGALVQNGQATALATVQQLDPIYVDVTQSSNDFLRLKQELANGTLKQENGKAKVSLITSDGIKFPQDGTLEFSDVTVDQTTGSITLRAIFPNPDHTLLPGMFVRARLEEGLNPNAILVPQQGVTRTPRGDATVLVVGADDKVETRPIVASQAIGDKWLVTEGLKAGDRVVISGLQKVRPGVQVKAQEVTADNNQQAASGAQPEQSKS

SEQ ID NO: 41 - DNA, 3150 bp, Escherichia coli K12
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccccggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggcgttatcaacaccgatggcaccatgacgcaggaggatatctccgactacgtggcggcgaatatgaaagatgccatcagccgtacgtcgggcgtgggtgatgttcagttgttcggttcacagtacgcgatgcgtatctggatgaacccgaatgagctgaacaaattccagctaacgccggttgatgtcattaccgccatcaaagcgcagaacgcccaggttgcggcgggtcagctcggtggtacgccgccggtgaaaggccaacagcttaacgcctctattattgctcagacgcgtctgacctctactgaagagttcggcaaaatcctgctgaaagtgaatcaggatggttcccgcgtgctgctgcgtgacgtcgcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccatacgacaccacgccgttcgtgaaaatctctattcacgaagtggttaaaacgctggtcgaagcgatcatcctcgtgttcctggttatgtatctgttcctgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgacgccatcgttgtggtagaaaacgttgagcgtgttatggcggaagaaggtttgccgccaaaagaagctacccgtaagtcgatggggcagattcagggcgctctggtcggtatcgcgatggtactgtcggcggtattcgtaccgatggccttctttggcggttctactggtgctatctatcgtcagttctctattaccattgtttcagcaatggcgctgtcggtactggtggcgttgatcctgactccagctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttacctggtgctgtatctgatcatcgtggtcggcatggcctatctgttcgtgcgtctgccaagctccttcttgccagatgaggaccagggcgtgtttatgaccatggttcagctgccagcaggtgcaacgcaggaacgtacacagaaagtgctcaatgaggtaacgcattactatctgaccaaagaaaagaacaacgttgagtcggtgttcgccgttaacggcttcggctttgcgggacgtggtcagaataccggtattgcgttcgtttccttgaaggactgggccgatcgtccgggcgaagaaaacaaagttgaagcgattaccatgcgtgcaacacgcgctttctcgcaaatcaaagatgcgatggttttcgcctttaacctgcccgcaatcgtggaactgggtactgcaaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggcagctatgtgaacgactttatcgaccgcggtcgtgtgaagaaagtttatgtcatgtcagaagcgaaataccgtatgctgccggatgatatcggcgactggtatgttcgtgctgctgatggtcagatggtgccattctcggcgttctcctcttctcgttgggagtacggttcgccgcgtctggaacgttacaacggcctgccatccatggaaatcttaggccaggcggcaccgggtaaaagtaccggtgaagcaatggagctgatggaacaactggcgagcaaactgcctaccggtgttggctatgactggacggggatgtcctatcaggaacgtctctccggcaaccaggcaccttcactgtacgcgatttcgttgattgtcgtgttcctgtgtctggcggcgctgtacgagagctggtcgattccgttctccgttatgctggtcgttccgctgggggttatcggtgcgttgctggctgccaccttccgtggcctgaccaatgacgtttacttccaggtaggcctgctcacaaccattgggttgtcggcgaagaacgcgatccttatcgtcgaattcgccaaagacttgatggataaagaaggtaaaggtctgattgaagcgacgcttgatgcggtgcggatgcgtttacgtccgatcctgatgacctcgctggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 42 - protein, 1049 aa, Escherichia coli K12 [sequence omitted]

SEQ ID NO: 43 - DNA, 1194 bp, Escherichia coli o157h7
atgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcgacatacgacagtgcgaaaggtgatctggcgaaagcccaggctgcagccaatatcgcgcaattgacggtgaatcgttatcagaaactgctcggtactcagtacatcagtaagcaagagtacgatcaggctctggctgatgcgcaacaggcgaatgctgcggtaactgcggcgaaagctgccgttgaaactgcgcggatcaatctggcttacaccaaagtcacctctccgattagcggtcgcattggtaagtcgaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcgctgatcaccagtgacggcattaagttcccgcaggacggtacgctggaattctctgacgttaccgttgatcagaccactgggtctatcaccctacgcgctatcttcccgaacccggatcacactctgctgccgggtatgttcgtgcgcgcacgtctggaagaagggcttaatccaaacgctattttagtcccgcaacagggcgtaacccgtacgccgcgtggcgatgccaccgtactggtggttggcgcggatgacaaagtggaaacccgtccgatcgttgcaagccaggctattggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtcttaa

SEQ ID NO: 44 - protein, 397 aa, Escherichia coli o157h7; identical to SEQ ID NO: 40

SEQ ID NO: 45 - DNA, 1230 bp, Escherichia coli CFT073
ttgaccaatttgaaatcggacactcgaggtttacatatgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcggcatacgacagtgcgaaaggtgatctggcgaaagcccaggctgcagccaatatcgcgcaattgacggtgaatcgttatcagaaattgctcggtactcagtacatcagtaagcaagagtacgatcaggctctggctgatgcgcaacaggcgaatgctgcggtaactgcggcgaaagctgccgttgaaactgcgcgaatcaatctggcttacaccaaagttacctctccgattagtggtcgcattggtaagtcaaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcgctgatcaccagtgacggcattaagttcccgcaggacggtacgctggaattctctgacgttaccgttgatcagaccactgggtctatcaccctacgcgctatcttcccgaacccggatcacactctgctgccgggtatgttcgtgcgtgcacgtctggaagaagggcttaatccaaacgctattttagtcccgcaacagggcgtaacccgtacgccgcgtggcgatgccaccgtactggtggttggcgcggatgacaaagtggaaacccgtccgatcgttgcaagccaggctatcggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtcttaa

SEQ ID NO: 46 - protein, 409 aa, Escherichia coli CFT073
MTNLKSDTRGLHMNKNRGFTPLAVVLMLSGSLALTGCDDKQAQQGGQQMPAVGVVTVKTEPLQITTELPGRTSAYRIAEVRPQVSGIILKRNFKEGSDIEAGVSLYQIDPATYQAAYDSAKGDLAKAQAAANIAQLTVNRYQKLLGTQYISKQEYDQALADAQQANAAVTAAKAAVETARINLAYTKVTSPISGRIGKSNVTEGALVQNGQATALATVQQLDPIYVDVTQSSNDFLRLKQELANGTLKQENGKAKVSLITSDGIKFPQDGTLEFSDVTVDQTTGSITLRAIFPNPDHTLLPGMFVRARLEEGLNPNAILVPQQGVTRTPRGDATVLVVGADDKVETRPIVASQAIGDKWLVTEGLKAGDRVVISGLQKVRPGVQVKAQEVTADNNQQAASGAQPEQSKS

SEQ ID NO: 47 - DNA, 1230 bp, Escherichia coli UT189
ttgaccaatttgaaatcggacactcgaggtttacatatgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcggcatacgacagtgcgaaaggtgatctggcgaaagcccaggctgcagccaatatcgcgcaattgacggtgaatcgttatcagaaattgctcggtactcagtacatcagtaagcaagagtacgatcaggctctggctgatgcgcaacaggcgaatgctgcggtaactgcggcgaaagctgccgttgaaactgcgcgaatcaatctggcttacaccaaagtcacctctccgattagtggtcgcattggtaagtcaaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcgctgatcaccagtgacggcattaagttcccgcaggacggtacgctggaattctctgacgttaccgttgatcagaccactgggtctatcaccctacgcgctatcttcccgaacccggatcacactctgctgccaggtatgttcgtgcgtgcacgtctggaagaagggcttaatccaaacgctattttagtcccgcaacagggcgtaacccgtacgccgcgtggcgatgccaccgtactggtggttggcgcggatgacaaagtggaaacccgtccgatcgttgcaagccaggctatcggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtcttaa

SEQ ID NO: 48 - protein, 409 aa, Escherichia coli UT189; identical to SEQ ID NO: 46

SEQ ID NO: 49 - DNA, 3150 bp, Escherichia coli o17h7
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccccggcgctgatgcgaaaacagtgcaggacacggtgacacaggttatcgaacagaatatgaacggtatcgataacctgatgtacatgtcctctaacagtgactccacgggtaccgtacagatcaccctgacctttgagtctggtactgatgcggatatcgcgcaggttcaggtgcagaacaaactgcagctggcgatgccgttgctgccgcaagaagttcagcagcaaggggtgagcattgagaaatcatccagcagcttcctgatggttgtcggcgttatcaacaccgatggcaccatgacgcaggaggatatctccgactacgtggcggcgaatatgaaagatgccatcagccgtacgtctggcgtgggtgacgttcagttgttcggttcacagtacgcgatgcgtatctggatgaacccgaatgaactgaacaaattccagctaacgccggttgatgttattaccgccatcaaagcgcagaacgcccaggttgcggcgggccagctcggtggtacaccgccggtgaaaggccaacagcttaacgcctctattattgctcagacgcgtctgacctctactgaagagttcggcaaaatcctgctgaaagtgaatcaggatggttcccgcgtactgctgcgtgatgtggcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccgtacgacaccacgccgttcgtgaaaatctctattcacgaagtggttaaaacgctggtcgaagcgatcatcctcgtgttcctggttatgtatctgttcctgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgacgctatcgttgtggtagaaaacgttgagcgtgtgatggcggaagaaggtttgccgccaaaagaagccacccgtaagtcgatggggcagattcagggcgctctggtcggtatcgcgatggtactgtcggcggtattcgtaccgatggccttctttggcggttctactggggcaatttatcgtcagttctctattaccattgtttcagcaatggcgctgtcggtactggtggcgttgatcctgactccagctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttatctggtgctgtatctgatcatcgtggtcggcatggcctatctgtttgtgcgtctgccaagctccttcttgccagatgaggaccagggcgtatttatgaccatggttcagctgccagcaggtgcaacgcaggaacgtacgcagaaagtgctcaatgaggtaacgcattactatctgaccaaagaaaagaacaacgttgagtcggtgttcgccgttaacggcttcggctttgcgggacgtggtcagaataccggtattgcgttcgtttccttgaaggactgggccgatcgtccgggtgaagaaaacaaagttgaagcgattaccatgcgtgcaacacgcgctttctcgcaaatcaaagatgcgatggttttcgcctttaacctgcccgcaatcgtggaactgggtaccgccaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggcagctatgtgaacgactttatcgaccgcggtcgtgtgaagaaagtttacgtcatgtcagaagcgaaataccgtatgctgccggatgatatcggcgactggtatgttcgtgctgctgatggtcagatggtgccattctcggcgttctcctcttctcgttgggagtacggttcgccgcgtctggaacgttacaacggcctgccatccatggaaatcttaggccaggcggcaccgggtaaaagtaccggtgaagcaatggagctgatggaacaactggcgagcaaactgcctaccggtgttggctatgactggacggggatgtcctatcaggaacgtctctccggcaaccaggcaccttcactgtacgcgatttcgttgattgtcgtgttcctgtgtctggcggcgctgtacgagagctggtcgattccgttctccgttatgctggtcgttccgctgggggttatcggtgcgttgctggctgccaccttccgtggcctgaccaatgacgtttacttccaggtaggcctgctcacaaccattgggttgtcggcgaagaacgcgatacttatcgtcgaattcgccaaagacttgatggataaagaaggtaaaggtctgattgaagcgacgcttgatgcggtgcggatgcgtttacgtccgatcctgatgacctcgctggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacaaccatactgtcgatcatcattga

SEQ ID NO: 50 - protein, 1049 aa, Escherichia coli o157h7
MPNFFIDRPIFAWVIAIIIMLAGGLAILKLPVAQYPTIAPPAVTISASYPGADAKTVQDTVTQVIEQNMNGIDNLMYMSSNSDSTGTVQITLTFESGTDADIAQVQVQNKLQLAMPLLPQEVQQQGVSIEKSSSSFLMVVGVINTDGTMTQEDISDYVAANMKDAISRTSGVGDVQLFGSQYAMRIWMNPNELNKFQLTPVDVITAIKAQNAQVAAGQLGGTPPVKGQQLNASIIAQTRLTSTEEFGKILLKVNQDGSRVLLRDVAKIELGGENYDIIAEFNGQPASGLGIKLATGANALDTAAAIRAELAKMEPFFPSGLKIVYPYDTTPFVKISIHEVVKTLVEAIILVFLVMYLFLQNFRATLIPTIAVPVVLLGTFAVLAAFGFSINTLTMFGMVLAIGLLVDDAIVVVENVERVMAEEGLPPKEATRKSMGQIQGALVGIAMVLSAVFVPMAFFGGSTGAIYRQFSITIVSAMALSVLVALILTPALCATMLKPIAKGDHGEGKKGFFGWFNRMFEKSTHHYTDSVGGILRSTGRYLVLYLIIVVGMAYLFVRLPSSFLPDEDQGVFMTMVQLPAGATQERTQKVLNEVTHYYLTKEKNNVESVFAVNGFGFAGRGQNTGIAFVSLKDWADRPGEENKVEAITMRATRAFSQIKDAMVFAFNLPAIVELGTATGFDFELIDQAGLGHEKLTQARNQLLAEAAKHPDMLTSVRPNGLEDTPQFKIDIDQEKAQALGVSINDINTTLGAAWGGSYVNDFIDRGRVKKVYVMSEAKYRMLPDDIGDWYVRAADGQMVPFSAFSSSRWEYGSPRLERYNGLPSMEILGQAAPGKSTGEAMELMEQLASKLPTGVGYDWTGMSYQERLSGNQAPSLYAISLIVVFLCLAALYESWSIPFSVMLVVPLGVIGALLAATFRGLTNDVYFQVGLLTTIGLSAKNAILIVEFAKDLMDKEGKGLIEATLDAVRMRLRPILMTSLAFILGVMPLVISTGAGSGAQNAVGTGVMGGMVTATVLAIFFVPVFFVVVRRRFSRKNEDIEHNHTVDHH

SEQ ID NO: 51 - DNA, 3150 bp, Escherichia coli CFT0731
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccctggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggggttatcaacaccgatggcactatgacgcaggaggatatctctgactacgtggcagcgaatatgaaagatgccatcagccgtacgtcgggcgtgggtgacgttcagttgttcggttcacagtacgcgatgcgtatctggatgaacccgaatgaactgaacaaattccagctaacgccggttgatgtcattaccgccatcaaagcacagaacgctcaggttgcagctggtcagctcggtggtacgccgccggtgaaaggccaacagcttaacgcctctattattgctcagacgcgtctgacctctactgaagagttcggcaaaatcctgctgaaagtgaatcaggatggttcccgcgtgctactgcgtgatgtggcgaaaattgagctgggtggtgagaactacgacatcatcgcagagtttaacggccaaccggcttccggtctggggatcaagctggcgaccggtgcgaacgcgctggataccgctgcggcaatccgtgctgaactggcgaagatggaaccgttcttcccgtcgggtctgaaaattgtttacccgtatgacaccacaccgttcgtgaaaatctctattcacgaagtggttaaaacgctggtcgaagcgatcatcctcgtgttcctggtaatgtatctgttcttgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgatgccatcgttgtggtagaaaacgttgagcgtgtgatggcggaagaaggtttgccgccaaaagaagccacccgtaagtcgatggggcagattcagggcgctctggtcggtatcgcgatggtactgtcggcggtattcgtaccgatggccttctttggcggttctactggtgctatctatcgtcagttctctattaccattgtttcagcaatggcgctgtcggtactggtggcgttgatcctgactccggctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttacctggtgctgtatctgatcatcgtggtcggtatggcctatctgttcgtgcgtctgccaagctccttcttgccagatgaggaccagggcgtatttatgaccatggttcagctgccagcaggtgcaacgcaggaacgtacgcagaaagtgctcaatgaggtaacgaattactatctgaccaaagaaaagaacaacgttgagtcggtgttcgccgttaacggcttcggctttgcgggacgtggtcagaataccggtattgcgttcgtttccttgaaggactgggccgatcgtccgggcgaagaaaacaaagttgaagcgattaccatgcgtgcaacacgtgctttctcgcaaatcaaagatgcgatggttttcgcctttaacctgcccgcaatcgtggaactgggtaccgcaaccggctttgactttgagctgattgaccaggcaggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggtagctatgtgaacgactttatcgaccgcggtcgtgtgaagaaagtttacgtcatgtcagaagcgaaataccgtatgctgccggatgatatcggcgactggtatgttcgtgctgctgatggtcagatggtgccgttctcggcgttctcctcttctcgttgggagtacggttcgccgcgtctggaacgttacaacggcctgccatctatggaaatcttaggccaggcggcaccgggtaaaagtaccggtgaagcaatggagctgatggaacaactggcgagcaaactgcctaccggtgttggctatgactggacgggaatgtcctatcaggaacgtctctccggcaaccaggcaccttcactgtacgcgatttcgttgattgtcgtgttcctgtgtctggctgcgctgtacgagagctggtcgattccgttctccgttatgctagtcgttccgctgggggttatcggtgcgttgctggctgccaccttccgtggcctgaccaatgacgtttacttccaggtaggcctgctcacaaccattggtttgtcggcgaagaacgcgatacttatcgtcgaattcgccaaagacttgatggataaagaaggtaaaggtctgattgaagcgacgcttgatgcggtgcggatgcgtttacgcccaatcctgatgacctcgttggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 52 - protein, 1049 aa, Escherichia coli CFT073
MPNFFIDRPIFAWVIAIIIMLAGGLAILKLPVAQYPTIAPPAVTISASYPGADAKTVQDTVTQVIEQNMNGIDNLMYMSSNSDSTGTVQITLTFESGTDADIAQVQVQNKLQLAMPLLPQEVQQQGVSVEKSSSSFLMVVGVINTDGTMTQEDISDYVAANMKDAISRTSGVGDVQLFGSQYAMRIWMNPNELNKFQLTPVDVITAIKAQNAQVAAGQLGGTPPVKGQQLNASIIAQTRLTSTEEFGKILLKVNQDGSRVLLRDVAKIELGGENYDIIAEFNGQPASGLGIKLATGANALDTAAAIRAELAKMEPFFPSGLKIVYPYDTTPFVKISIHEVVKTLVEAIILVFLVMYLFLQNFRATLIPTIAVPVVLLGTFAVLAAFGFSINTLTMFGMVLAIGLLVDDAIVVVENVERVMAEEGLPPKEATRKSMGQIQGALVGIAMVLSAVFVPMAFFGGSTGAIYRQFSITIVSAMALSVLVALILTPALCATMLKPIAKGDHGEGKKGFFGWFNRMFEKSTHHYTDSVGGILRSTGRYLVLYLIIVVGMAYLFVRLPSSFLPDEDQGVFMTMVQLPAGATQERTQKVLNEVTNYYLTKEKNNVESVFAVNGFGFAGRGQNTGIAFVSLKDWADRPGEENKVEAITMRATRAFSQIKDAMVFAFNLPAIVELGTATGFDFELIDQAGLGHEKLTQARNQLLAEAAKHPDMLTSVRPNGLEDTPQFKIDIDQEKAQALGVSINDINTTLGAAWGGSYVNDFIDRGRVKKVYVMSEAKYRMLPDDIGDWYVRAADGQMVPFSAFSSSRWEYGSPRLERYNGLPSMEILGQAAPGKSTGEAMELMEQLASKLPTGVGYDWTGMSYQERLSGNQAPSLYAISLIVVFLCLAALYESWSIPFSVMLVVPLGVIGALLAATFRGLTNDVYFQVGLLTTIGLSAKNAILIVEFAKDLMDKEGKGLIEATLDAVRMRLRPILMTSLAFILGVMPLVISTGAGSGAQNAVGTGVMGGMVTATVLAIFFVPVFFVVVRRRFSRKNEDIEHSHTVDHH

SEQ ID NO: 53 - DNA, 3150 bp, Escherichia coli UTI89
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccctggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggggttatcaacaccgatggcactatgacgcaggaggatatctctgactacgtggcagcgaatatgaaagatgccatcagccgtacgtcgggcgtgggtgacgttcagttgttcggttcacagtacgcgatgcgtatctggatgaacccgaatgaactgaacaaattccagctaacgccggttgatgtcattaccgccatcaaagcacagaacgctcaggttgcagctggtcagctcggtggtacgccgccggtgaaaggccaacagcttaacgcctctattattgctcagacgcgtctgacctctactgaagagttcggcaaaatcctgctgaaagtgaatcaggatggttcccgcgtgctactgcgtgatgtggcgaaaattgagctgggtggtgagaactacgacatcatcgcagagtttaacggccaaccggcttccggtctggggatcaagctggcgaccggtgcgaacgcgctggataccgctgcggcaatccgtgctgaactggcgaagatggaaccgttcttcccgtcgggtctgaaaattgtttacccgtatgacaccacaccgttcgtgaaaatctctattcacgaagtggtaaaaacgctggtcgaagcgatcatcctcgtgttcctggtaatgtatctgttcctgcagaacttccgcgcgacgttgattccgactattgctgtaccggtggtattgctggggacatttgccgtccttgccgcctttggcttctcgataaacacactaacgatgttcgggatggtactcgccatcggcctgttggtggatgacgccatcgttgtagtagaaaacgttgagcgtgtaatggcagaagaaggtctgccaccgaaagaagctacgcgtaagtcgatggggcagattcagggcgcgctggtgggtatcgcgatggtactgtcagcggtattcgtaccgatggccttcttcggcggttcgactggggcaatttatcgtcagttctccattaccattgtttcggcaatggcgctgtcggtactggtggcgttgatcctgactccggcactctgtgcaaccatgctgaaaccgattgccaaaggcgatcacggcgaaggtaaaaaaggcttcttcggctggtttaaccgcatgttcgagaagagcacgcaccactacaccgacagcgtaggcggtattctgcgcagtacagggcgttacctggtgctgtatctgatcatcgtggtcggcatggcctatctgttcgtgcgtctgccaagctccttcttgccagatgaagaccagggcgtatttatgaccatggttcagctgccagcaggtgcaacgcaggaacgtacgcagaaagtgctcaatgaggtaacgcattactatctgaccaaagaaaagaacaacgttgagtcggtgttcgccgttaacggcttcggctttgcgggacgtggtcagaatacaggtattgcgttcgtttccttgaaggactgggccgatcgtccgggcgaagaaaacaaagttgaagcgattaccatgcgtgcaacacgtgctttctcgcaaatcaaagatgcgatggttttcgcctttaacctgcccgcaatcgtggaactgggtaccgcaaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggtagctatgtgaacgactttatcgaccgcggtcgtgtgaagaaagtttacgtcatgtcagaagcgaaataccgtatgctgccggatgatatcggcgactggtatgttcgtgctgctgatggtcagatggtgccgttctcggcgttctcctcttctcgttgggagtacggttcgccgcgtctggaacgttacaacggcctgccatctatggaaatcttaggccaggcggcaccgggtaaaagtaccggtgaagcaatggagctgatggaacaactggcgagcaaactgcctaccggtgttggctatgactggacgggaatgtcctatcaggaacgtctctccggcaaccaggcgccttcactgtacgcgatttcgttgattgtcgtgttcctgtgtctggctgcgctgtacgagagctggtcgattccgttctccgttatgctggtcgttccgctgggggttatcggtgcgttgctggctgccaccttccgtggcctgaccaatgacgtttacttccaggtaggcctgctcacaaccattggtttgtcggcgaagaacgcgatacttatcgtcgaattcgccaaagacttgatggataaagaaggtaaaggtctgattgaagcgacgcttgatgcggtgcggatgcgtttacgcccaatcctgatgacctcgttggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 54 - protein, 1049 aa, Escherichia coli UTI89 [sequence omitted]

SEQ ID NO: 55 - DNA, 2109 bp, Escherichia coli K12
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 56 - protein, 702 aa, Escherichia coli K12
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 57 - DNA, 2109 bp, Escherichia coli o157h7
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttacagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgaaaagcatctttattcgatttactgtaaaatggtgctcaaagagcagcgttttcactcaatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtcgttgcacacctcgatgattggcccgcacggcgtgccggttgaggtccagatccgtaccgaagatatggatcagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggccatgcctgcgtgggcgcacgcgtcgaccgccagccttacccgctgtcgcagccgcttactagcggtcagaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctcaatgaaatcccgcaggaaaatattcagcgcgagctggatcgcatgaagctggcaacgcttgacgatctgctggcagaaatcggccttggtaacgcaatgagcgtggtggtcgcgaaaaatctacaacatggggacgcctccattccaccggcgactcaaagccacgggcatctgccgatcaaaggcgctgatggcgtgctgatcacctttgcgaaatgctgccgccccattcctggcgacccgattatcgcccacgtcagccccggtaaaggtctggtgatccaccatgaatcctgccgtaacatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacctcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 58 - protein, 702 aa, Escherichia coli o157h7; identical to SEQ ID NO: 56

SEQ ID NO: 59 - DNA, 2109 bp, Escherichia coli CFT073
ttgtatctgtttgaaagcctgaatcagctgattcaaaactacctgccggaagaccaaatcaagcgtctgcggcaggcgtatctcgttgcacgtgatgctcacgaggggcaaacacgttcaagcggtgaaccctatatcacgcatccggtggcggttgcctgcattctggccgagatgaaactcgactatgaaacgctgatggcggcgctgctgcatgatgtgattgaagatacacccgccacctaccaggacatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcattctcatcaaacttgccgaccgtacccacaacatgcgcacgctgggctcacttcgcccggacaaacgtcgccgcatcgcccgtgaaactctcgaaatatacagcccgctggcgcaccgtttaggtatccaccacattaaaaccgaactcgaggagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgaaaaacatctttattcgatttactgcaaaatggtgctcaaagagcagcgttttcactcaatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgattggcccgcacggcgtgccggttgaggtccagatccgtaccgaagatatggatcagatggcggagatgggtgttgccgcgcactgggcttataaagagcacggcgaaaccagcactaccgcgcaaatacgcgcccagcgctggatgcaaagcctgctggagctgcaacagagtgccggtagctcgtttgaatttatcgagagcgttaaatccgatctcttcccggatgagatttacgttttcacaccggaagggcgcattgtcgaactgcctgccggtgcaacgcccgtcgacttcgcttatgcggtgcataccgatatcggccatgcctgcgtgggcgcacgcgtcgaccgccagccttacccgctgtcgcagccgcttactagcggtcaaaccgttgaaatcattaccgcaccgggtgctcgcccgaatgccgcgtggctgaactttgtcgtcagctcgaaagcgcgcgccaaaattcgtcagttgctgaaaaacctcaagcgtgatgattctgtaagcctgggccgtcgtctgctcaaccatgcgttgggtggtagccgtaagctcaatgaaatcccgcaggaaaatattcagcgcgagctggaccgcatgaagctggcaacgcttgacgatctgctggcagaaatcggccttggtaacgcaatgagcgtggtggtcgcgaaaaatctgcaacatggggacgcctccattccaccggcaacccaaagtcacggacatctgcccattaaaggtgccgatggcgtgctgatcacctttgcgaaatgctgccgcccaattcctggcgacccgattatcgcccacgtcagccccggtaaaggtctggtgatccaccatgaatcctgccgtaacatccgtggctaccagaaagagccagagaagtttatggctgttgaatgggataaagagacggcgcaggaattcatcaccgaaatcaaggtggagatgttcaatcatcagggcgcgctggcaaacctgacggcggcaattaacaccacgacctcgaatattcaaagtttgaatacggaagagaaagatggtcgcgtctatagcgcctttattcgtctgaccgcccgtgaccgtgtgcatctggcgaatatcatgcgcaaaatccgcgtgatgccagacgtgattaaagtcacccgaaaccgaaattaa

SEQ ID NO: 60 - protein, 702 aa, Escherichia coli CFT073
MYLFESLNQLIQNYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 61 - DNA, 2109 bp, Escherichia coli UTI89
ttgtatctgtttgaaagcctgaatcagctgattcaaaactacctgccggaagaccaaatcaagcgtctgcggcaggcgtatctcgttgcacgtgatgctcacgaggggcaaacacgttcaagcggtgaaccctatatcacgcacccggtggcggttgcctgcattctggccgagatgaaactcgactatgaaacgctgatggcggcgctgctgcatgatgtgattgaagatacacccgccacctaccaggacatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcattctcatcaaacttgccgaccgtacccataacatgcgcacgctgggctcacttcgcccggacaaacgtcgccgcatcgcccgtgaaactctcgaaatatacagcccgctggcgcaccgtttaggtatccaccacattaaaaccgaactcgaggagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgaaaaacatctttattcgatttactgcaaaatggtgctcaaagagcagcgttttcactcaatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgattggcccgcacggcgtgccggttgaggtccagatccgtaccgaagatatggatcagatggcggagatgggtgttgccgcgcactgggcttataaagagcacggcgaaaccagcactaccgcgcaaatacgcgcccagcgctggatgcaaagcctgctggagctgcaacagagtgccggtagctcgtttgaatttatcgagagcgttaaatccgatctcttcccggatgagatttacgttttcacaccagaagggcgcattgtcgaactgcctgccggtgcaacgcccgtcgacttcgcttatgcggtgcataccgatatcggccatgcctgcgtgggcgcacgcgtcgaccgccagccttacccgctgtcgcagccgcttactagcggtcaaaccgttgaaatcattaccgcaccgggtgctcgcccgaatgccgcgtggctgaactttgtcgtcagctcgaaagcgcgcgccaaaattcgtcagttgctgaaaaacctcaagcgtgatgattctgtaagcctgggccgtcgtctgctcaaccatgcgttgggtggtagccgtaagctcaatgaaatcccgcaggaaaatattcagcgcgagctggaccgcatgaagctggcaacgcttgacgatctgctggcagaaatcggccttggtaacgcaatgagcgtggtggtcgcgaaaaatctgcaacatggggacgcctccattccaccggcaacccaaagtcacggacatctgcccattaaaggtgccgatggcgtgctgatcacctttgcgaaatgctgccgcccaattcctggcgacccgattatcgcccacgtcagccccggtaaaggtctggtgatccaccatgaatcctgccgtaacatccgtggctaccagaaagagccagagaagtttatggctgttgaatgggataaagagacggcgcaggaattcatcaccgaaatcaaggtggagatgttcaatcatcagggcgcgctggcaaacctgacggcggcaattaacaccacgacctcgaatattcaaagtttgaatacggaagagaaagatggtcgcgtctatagcgcctttattcgtctgaccgcccgtgaccgtgtgcatctggcgaatatcatgcgcaaaatccgcgtgatgccagacgtgattaaagtcacccgaaaccgaaattaa

SEQ ID NO: 62 - protein, 702 aa, Escherichia coli UTI89; identical to SEQ ID NO: 60

SEQ ID NO: 63 - DNA, 2235 bp, Escherichia coli K12
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgcttttccctctggcggatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtagtcatcaaactggcggagcgtattgctcatctgcgcgaagtaaaagatgcgccggaagatgaacgtgtactggcggcaaaagagtgtaccaacatctacgcaccgctggctaaccgtctcggaatcggacaactgaaatgggaactggaagattactgcttccgttacctccatccaaccgaatacaaacgaattgccaaactgctgcatgaacggcgtctcgaccgcgaacactacatcgaagagttcgttggtcatctgcgcgctgagatgaaagctgaaggcgttaaagcggaagtgtatggtcgtccgaaacacatctacagcatctggcgtaaaatgcagaaaaagaacctcgcctttgatgagctgtttgatgtgcgtgcggtacgtattgtcgccgagcgtttacaggattgctatgccgcactggggatagtgcacactcactatcgccacctgccggatgagtttgacgattacgtcgctaacccgaaaccaaacggttatcagtctattcataccgtggttctggggccgggtggaaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgtagtcaggtctttgacgaccgggtgtacgtctttacgccgaaaggtgatgtcgttgatttgcctgcgggatcaacgccgctggacttcgcttaccacatccacagtgatgtcggacaccgctgcatcggggcaaaaattggcgggcgcattgtgccgttcacctaccagctgcagatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaatccttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaagcaacttcagcaaaaaagctacacgccgcaaaaccgcagtaaagataacggtcgcgtggtagtcgaaggtgttggcaacctgatgcaccacatcgcgcgctgctgccagccgattcctggagatgagattgtcggcttcattacccaggggcgcggtatttcagtacaccgcgccgattgcgaacaactggcggaactgcgctcccatgcgccagaacgcattgttgacgcggtatggggtgagagctactccgccggatattcgctggtggtccgcgtggtagctaatgatcgtagtgggttgttacgtgatatcacgaccattctcgccaacgagaaggtgaacgtgcttggcgttgccagccgtagcgacaccaaacagcaactggcgaccatcgacatgaccattgagatttacaacctgcaagtgctggggcgcgtgctgggtaaactcaaccaggtgccggatgttatcgacgcgcgtcggttgcacgggagttag

SEQ ID NO: 64 - protein, 744 aa, Escherichia coli K12
MVAVRSAHINKAGEFDPEKWIASLGITSQKSCECLAETWAYCLQQTQGHPDASLLLWRGVEMVEILSTLSMDIDTLRAALLFPLADANVVSEDVLRESVGKSVVNLIHGVRDMAAIRQLKATHTDSVSSEQVDNVRRMLLAMVDDFRCVVIKLAERIAHLREVKDAPEDERVLAAKECTNIYAPLANRLGIGQLKWELEDYCFRYLHPTEYKRIAKLLHERRLDREHYIEEFVGHLRAEMKAEGVKAEVYGRPKHIYSIWRKMQKKNLAFDELFDVRAVRIVAERLQDCYAALGIVHTHYRHLPDEFDDYVANPKPNGYQSIHTVVLGPGGKTVEIQIRTKQMHEDAELGVAAHWKYKEGAAAGGARSGHEDRIAWLRKLIAWQEEMADSGEMLDEVRSQVFDDRVYVFTPKGDVVDLPAGSTPLDFAYHIHSDVGHRCIGAKIGGRIVPFTYQLQMGDQIEIITQKQPNPSRDWLNPNLGYVTTSRGRSKIHAWFRKQDRDKNILAGRQILDDELEHLGISLKEAEKHLLPRYNFNDVDELLAAIGGGDIRLNQMVNFLQSQFNKPSAEEQDAAALKQLQQKSYTPQNRSKDNGRVVVEGVGNLMHHIARCCQPIPGDEIVGFITQGRGISVHRADCEQLAELRSHAPERIVDAVWGESYSAGYSLVVRVVANDRSGLLRDITTILANEKVNVLGVASRSDTKQQLATIDMTIEIYNLQVLGRVLGKLNQVPDVIDARRLHGS

SEQ ID NO: 65 - DNA, 2235 bp, Escherichia coli o157h7
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgctgttccctctggctgatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtggtcatcaaactggcggagcgtattgctcatctgcgtgaagtaaaagatgcgccggaagatgaacgcgtactggcggcaaaagagtgcaccaatatctacgcgccgttggcaaaccgtcttgggattgggcaactgaaatgggagctggaagattactgcttccgttatctccacccgaccgaatacaaacgcatcgcaaaactgttgcatgaacgccgtctcgaccgcgaacactatatcgaagagtttgtcggtcatctgcgcgctgagatgaaagctgaaggcgttaaagctgaagtgtatggtcgtccgaaacacatctacagcatctggcgcaaaatgcagaaaaagaacctcgccttcgatgagctgtttgatgtgcgtgcggtacgtattgtcgccgagcgtttacaggattgttatgccgcactggggatagtgcacactcactatcgccacctgccggatgagtttgacgattacgtcgctaacccgaaaccaaacggttatcagtctattcataccgtggttctggggccgggtggaaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgcagccaggtctttgacgaccgggtgtacgtctttacgcctaaaggtgatgtcgttgatttgcctgcgggatcaacgccgctggacttcgcttaccacatccacagtgatgtcggacaccgctgtatcggggcaaaaattggcgggcgcattgtgccgttcacctaccagctgcaaatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaatccttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaaacagcttcagcaaaaaagctacacgccgcaaaaccgcagtaaagataacggtcgtgtagtggttgaaggtgttggtaacctgatgcaccacatcgcgcgctgctgccagccgattcctggagatgagattgtcggcttcattacccagggacgcggtatttcagtacaccgcgccgattgcgaacaactggcggaactgcgctcccatgcgccagaacgcattgttgacgcggtatggggtgagagctactccgccggatattcgctggtggtccgcgtggtggctaatgatcgtagtgggttgttacgtgatatcacgaccattctcgccaacgagaaggtgaacgtgcttggcgttgccagccgtagcgacaccaaacagcaactggcgaccatcgacatgaccattgagatttacaacctgcaagtgctggggcgcgtgctgggtaaactcaaccaggtgccggatgttatcgacgcgcgtcggttgcacgggagttag

SEQ ID NO: 66 - protein, 744 aa, Escherichia coli o157h7; identical to SEQ ID NO: 64

SEQ ID NO: 67 - DNA, 2235 bp, Escherichia coli CFT073
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgctgttccctctggctgatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtggtcatcaaactggcggagcgtattgctcacctgcgcgaagtaaaagatgcgccggaagatgaacgcgtactggcggcaaaagagtgcaccaatatctacgcgccgttggcgaaccgtcttgggattgggcaactgaaatgggagctggaagattactgcttccgttatctgcacccgaccgaatacaaacgcatcgcaaaactgctgcatgaacgccgtctcgaccgcgaacactatatcgaagagtttgtcggccatctgcgcgctgagatgaaagctgaaggtgttaaagctgaagtgtatggtcgaccgaaacacatctacagcatctggcgcaaaatgcagaaaaagaacctcgccttcgatgagctgtttgatgtgcgtgcggtacgtattgtcgccgagcgtttacaggattgttatgccgcactggggatagtgcacactcactatcgccacctgccggatgagtttgacgattacgtcgctaacccgaaaccaaacggttatcagtccattcataccgtggttctggggccgggtggcaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgtagtcaggtctttgacgaccgagtgtacgtctttacgccgaaaggtgatgtcgttgatttgcctgcgggatcaacgccgctggacttcgcttaccacatccacagtgatgtcggacaccgctgcatcggggcaaaaattggcgggcgcattgtgccgttcacctaccagctgcagatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaatccttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaagcaacttcagcaaaaaagctacacgccgcaaaaccgcagtaaagataacggtcgtgtggtggttgaaggtgtcggtaacctgatgcaccacatcgcgcgctgctgtcagcctattcctggtgatgaaatagttggtttcattactcagggacgcggtatttcagtacaccgcgccgattgcgaacaactggcggaactgcgctcccatgcgccagaacgcattgttgacgcggtatggggtgagagctactccgccggatattcgctggtggtccgcgtggtggctaatgatcgtagtgggttgttacgtgatatcacgaccattctcgccaacgagaaggtgaacgtgcttggcgttgccagccgtagcgacaccaaacagcaactggcgaccatcgacatgaccattgagatttacaacctgcaagtgctgggccgcgtgctgggtaaactcaaccaggtaccggatgttatcgacgcgcgtcggttgcacgggagttaa

SEQ ID NO: 68 - protein, 744 aa, Escherichia coli CFT073; identical to SEQ ID NO: 64

SEQ ID NO: 69 - DNA, 2235 bp, Escherichia coli UTI89
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgctgttccctctggctgatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtggtcatcaaactggcggagcgtattgctcacctgcgcgaagtaaaagatgcgccggaagatgaacgcgtactggcggcaaaagagtgcaccaatatctacgcgccgttggcgaaccgtcttgggattgggcaactgaaatgggagctggaagattactgcttccgttatctccacccgaccgaatacaaacgcatcgcaaaactgctgcatgaacgccgtctcgaccgcgaacactatatcgaagagtttgtcggccatctgcgcgctgagatgaaagctgaaggcgttaaagctgaagtgtatggccgtccgaaacacatctacagtatctggcgcaaaatgcagaaaaagaacctcgccttcgatgagctgtttgatgtgcgtgcggtacgtattgtcgccgagcgtttacaggattgttatgccgcactggggatagtgcacactcactatcgccacctgccggatgagtttgacgattacgtcgctaacccgaaaccaaacggttatcagtccattcataccgtggttctggggccgggtggcaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgtagtcaggtctttgacgaccgagtgtacgtctttacgccgaaaggtgatgtcgttgatttgcctgcgggatcaacgccgctggacttcgcttaccacatccacagtgatgtcggacaccgctgcatcggggcaaaaattggcgggcgcattgtgccgttcacctaccagctgcagatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaattcttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaaacagcttcagcaaaaaagctacacgccgcaaaaccgcagtaaagataacggtcgtgtggtggtagaaggggtcggtaacctgatgcaccacatcgcgcgctgctgccagccgattcctggagatgagattgtcggcttcattacccaggggcgcggtatttcagtacaccgcgccgattgcgaacaattggcggaactgcgctcccatgcgccagaacgcattgttgacgcggtatggggcgagagctactccgccggatattcgctggtggtccgcgtggtggctaatgatcgtagtgggttgttacgtgatatcacgaccattctcgccaacgagaaggtgaacgtacttggcgttgccagccgtagcgacaccaaacagcaactggcgaccatcgacatgaccattgagatttacaacctgcaagtgctggggcgcgtgctgggtaaactcaaccaggtaccggatgttatcgacgcgcgtcggttgcacgggagttag

SEQ ID NO: 70 - protein, 744 aa, Escherichia coli UTI89; identical to SEQ ID NO: 64

SEQ ID NO: 71 - DNA, 300 bp, Escherichia coli
tttaaaatgccagtagattgcaccgcgcgtaacgccagctgcttttgcaatctcgcccagcgaggtggatgataccccctgctgtgagaaaagacgtagagccacatcgaggatgtgttggcgcgtttcttgcgcttcttgtttggtttttcgtgccatatgttcgtgaatttacaggcgttagatttacatacatttgtgaatgtatgtaccatagcacgacgataatataaacgcagcaatgggtttattaacttttgaccattgaccaatttgaaatcggacactcgaggtttacat

SEQ ID NO: 72 - DNA, 28 bp, artificial sequence
  primer
ctggtccacctacaacaaagctctcatc

SEQ ID NO: 73 - DNA, 34 bp, artificial sequence
  primer
cttgtgcaatgtaacatcagagattttgagacac